(19) United States
(12) Patent Application Publication
(10) Pub. No.: US 2007/0270339 A1
(43) Pub. Date: Nov. 22, 2007

(54) Use of GPCR54 Ligands for the Treatment of Infertility

(75) Inventors: Robert K. Campbell, Wrentham, MA (US); Xuliang Jiang, Briantree, MA (US); Shoji Nagatani, Tokyo (JP)

Correspondence Address: MARSHALL, GERSTEIN & BORUN LLP, 233 S. WACKER DRIVE, SUITE 6300, SEARS TOWER, CHICAGO, IL, 60606, US

(73) Assignee: SERONO, INC., ROCKLAND, MA (US)

(21) Appl. No.: 11/547,293

(22) PCT Filed: Apr. 19, 2005
(86) PCT No.: PCT/US05/13339

§ 371 Date: Jul. 17, 2007

Related U.S. Application Data
(60) Provisional application No. 60/564,756, filed on Apr. 23, 2004.

(51) Int. Cl. A61P 15/08; A61K 31/085; A61K 38/08; A61K 38/17; A61K 38/24

(52) U.S. Cl. 514/12; 514/15; 514/17; 514/2

(57) ABSTRACT

The present invention is directed to methods and compositions for the treatment of infertility.

### BACKGROUND

1. Field of the Invention

The present invention is generally directed to the treatment of reproductive disorders. More specifically, the invention involves the treatment of such disorders by administering compositions comprising Kiss-1-derived peptides.

2. Background of the Related Art

In 1998, the American Society for Reproductive Medicine estimated that there were 6.1 million couples with infertility problems in the United States. Numerous advances have been made in the area of assisted reproductive technologies (ART) in efforts to overcome infertility problems in these individuals by the use of high technology procedures to combine sperm and eggs. Most couples go through extensive infertility evaluation and treatment before being considered for ART therapies. ART includes in vitro fertilization techniques alone typically in combination with various hormonal and/or surgical interventions.

Treatment of infertility will often require ovulation induction (OI) and/or controlled ovarian hyperstimulation (COH). OI involves causing a single follicle to pass through the cycle through to ovulation as a single oocyte whereas COH is directed at harvesting multiple oocytes for use in various in vitro ART procedures (e.g., for in vitro fertilization). Both of these procedures rely on the use of two primary hormones which control ovary follicle development and ovulation in women, namely: follicle-stimulating hormone (FSH) and luteinizing hormone (LH).

Women with normal ovarian function ovulate sometime during the middle of each menstrual cycle. In each menstrual cycle, many follicles are recruited for the maturation of the oocytes. At the beginning of the approximately 28-day menstrual cycle the follicles are in the primordial form, i.e., an oocyte surrounded by a single layer of cells. As follicular growth and maturation is activated by FSH, multiple layers of granulosa cells form around the initial single layer of cells, a process that continues through to midcycle. These granulosa cells are responsible for nourishing the oocyte and for the production and release of estrogen. FSH, produced by the pituitary induces aromatase activity in the granulosa cells thereby increasing the production of estrogen. Thus, concurrent with the maturation of a follicle there is an increase in estrogen production in the early part of the 28-day menstrual cycle. The follicle also contains receptors for the second pituitary gonadotropin, LH. As the follicle continues to grow and mature by mid-cycle (approx. day 14), a space (antrum) develops inside the granulosa cells. At mid-cycle a surge of LH production acts on LH receptors to cause the follicle to rupture and release the oocyte which travels down the fallopian tube and, which may subsequently be fertilized.

The normal ovulating woman recruits approx. 300 immature oocytes for each menstrual cycle. During a normal cycle, all but one follicle will regress (atresia), and a single dominant follicle will emerge and go on to release an oocyte. In vitro fertilization (IVF) of human oocytes, which is now a commonly used treatment for female and male subfertility, is based on retrieval of mature human oocytes followed by fertilization of the mature oocytes with spermatozoa. The recruitment of human mature oocytes is accomplished by hormone treatment regimens. For example, standard IVF treatment protocols include a long phase of hormone stimulation of the female patient, e.g. 30 days. This protocol is initiated by suppressing the patient's own FSH and LH by gonadotropin releasing hormone GnRH or an analog of GnRH, and is followed by injections of exogenous gonadotropins, e.g. FSH and/or LH, in order to ensure development of multiple preovulatory follicles. At an appropriate stage of follicular growth, multiple oocytes are harvested by aspiration immediately before ovulation. The aspirated oocyte is subsequently fertilized in vitro and cultured, typically for three days before transfer of the resulting embryo into the uterus at the 4-8 cell stage.

In women who do not ovulate naturally, an initial aim of intervention is to induce ovulation of at least one oocyte. Several medicaments are available and in use for artificially triggering and sustaining the development of follicles. These medicaments include clomiphene citrate (Clomid®, Serophene®) or Letrozole (Femera®), or alternatively, in the event that the patient is non-responsive to clomiphene, then injectable hormones, such as human menopausal gonadotropin (hMG) (Repronex®, Pergonal®, Humegon®), FSH (Gonal-F®, Follistim®, Bravelle®), human chorionic gonadotropin (hCG; Profasi®, Pregnyl®, Novarel®, Ovudrel®) to trigger the release of the ovum administered either alone or in combination with a GnRH antagonist such as Lupron®, Synarel®, Antigon®, or Cetrotide® to prevent the natural LH surge from occurring.

Clomiphene citrate is often the first drug used in fertility treatments as it is relatively inexpensive and is available in pill form and 60% to 80% of women treated with this medicament will ovulate. Clomiphene acts as an anti-estrogen on the central nervous system and causes an increase in the pulse frequency and concentration of FSH released by the pituitary and also causing stimulation of LH burst thereby giving a moderate gonadotropin stimulus to the ovary. Clomiphene is administered over a period of five days early in the cycle at typical doses that start at 50 mg/day and may be increased in later cycles up to 200 mg.

However, clomiphene administration has significant side effects. For example, there is a well-characterized possibility of multiple pregnancy. Further, it has been suggested that prolonged use of clomiphene may increase the risk of ovarian cancer. In addition, while clomiphene causes an increase in the rate of ovulation, the rate of pregnancy in clomiphene-stimulated women is surprisingly low. A variety of reasons have been suggested for this discrepancy between the rate of ovulation and the rate of pregnancy. Clomiphene appears to have an anti-estrogenic effect on the endometrium and seems to cause a decrease in the uterine lining. Secondly, the anti-estrogenic effect of clomiphene on the cervix is to decrease the amount of mucus produced from the cervix. Further, clomiphene administration appears to cause a decrease in uterine blood flow, impairs placental protein 14 production and may have detrimental effects on the oocyte. (see Tarlatziz & Grimbais, Hum. Reprod., 13:9 2356-2358, 1998; Out et al., Hum. Reprod., 13:9 2358-2361, 1998; Dickey et al., Hum. Reprod., 13:9 2361-2365, 1998). Finally, as clomiphene is an artificial chemical rather than a “natural” ovarian stimulant derived from a naturally occurring protein or other molecule, there are concerns that the side effects of such chemical intervention (hot flushes, mood swings, sleeplessness, dizziness, visual impairment) are too wide and varied for this intervention to be considered a safe stimulant. Indeed, it has been suggested that it is “highly unlikely that clomiphene citrate would be allowed to enter the market if registration were sought nowadays.” (see Out et al., Hum. Reprod., 13:9 2358-2361, 1998).

Thus, there are clear problems with the use of clomiphene-induced ovarian stimulation. Hormonal stimulation also has its drawbacks including risk of ovarian hyper stimulation syndrome (OHSS), weight gain, bloating, nausea, vomiting, the time involved with the monitoring process, and the unknown long-term cancer risks. Thus, there remains a need for new and effective methods for OI and/or COH.

### SUMMARY OF THE INVENTION

The present invention is directed to methods and compositions that for the ovarian stimulation and for replacing or augmenting existing methods for fertility treatment. In particular embodiments, the present invention contemplates methods for stimulating ovulation in an mammal comprising administering to the mammal a first composition comprising a Kiss-1 derived protein. In more particular embodiments, the Kiss-1 derived protein is a mature Kiss-1 protein comprising the sequence of SEQ ID NO:2. In other aspects, the method involves use of a Kiss-1 derived protein that is a peptide derived from a mature Kiss-1 protein. Preferably, the Kiss-1 derived protein is a Kiss-1 derived peptide that is able to activate a GPCR54 receptor. In specific embodiments, the Kiss-1 derived protein is a peptide that comprises an amino acid sequence of SEQ ID NO:7.

As described throughout the specification, numerous Kiss-1 peptides are encompassed herein. In specific preferred embodiments, the Kiss-1 derived protein is a peptide having the sequence of having the sequence of SEQ ID NO:3, an analog of a peptide of SEQ ID NO:3, a fragment of SEQ ID NO:3, or an analog of a fragment of SEQ ID NO:3. Variant, mutants, homologs and analogs are contemplated. In specific embodiments, the analog of SEQ ID NO:3 is a peptide that is a conservative variant of SEQ ID NO:3.

The Kiss-1 related peptide compositions of the present invention may be used in a combination with other compositions. For example, the method of stimulating ovulation may further comprise administering a second composition that stimulates ovulation. Compositions that stimulate ovulation are generally known to those of skill in the art and include hormonal and a chemical stimulants of ovulation. In exemplary embodiments, the chemical stimulant of ovulation is clomiphene citrate or Letrazole. Other agents that are analogs or variants of clomiphene citrate or Letrazole, or act through similar mechanisms of action also may be used in the methods described herein. The hormonal stimulant of ovulation may be any hormone used for such a purpose. Exemplary such hormones include gonadotropin hormones selected from the group consisting of human menopausal gonadotropin (hMG), follicle stimulating hormone (FSH), luteinizing hormone (LH), and human chorionic gonadotropin (hCG). In additional embodiments, the gonadotropin hormones may be combined with administration of a composition comprising GnRH antagonist. In particularly preferred embodiments, the gonadotropin is FSH.

The methods of the invention employing administration of FSH also may be further combined with administration of a non-FSH gonadotropin hormone. In particular embodiments, the non-FSH hormone is LH. The FSH and the LH may be administered in equal amounts, or may be administered in non-equal amounts. The FSH may be administered concurrently or separately.

In certain exemplary embodiments, the FSH is administered at a dosage range of from about 5 to 450 IU/day. In other embodiments, the FSH is administered at a dosage range of from about 5 to 75 IU/day. In specific aspects, the hormones may be administered through any route of administration employed for hormone treatment. More particularly, the gonadotropin hormone is administered by injection. The methods of the invention preferably involve treatment of a human.

A further aspect of the present invention encompasses a method of stimulating FSH production in a mammal comprising administering to the mammal a composition comprising a Kiss-1 derived protein. The Kiss-1 derived protein may be any protein that is derived from the mature Kiss-1 protein comprising the sequence of SEQ ID NO:2. The protein may be a peptide derived from a mature Kiss-1 protein. Preferably, the method involves use of a Kiss-1 derived peptide that is able to activate a G-protein coupled receptor 54. More particularly, the Kiss-1 derived protein is a peptide that comprises an amino acid sequence of SEQ ID NO:7. The Kiss-1 derived protein is preferably a peptide having the sequence of SEQ ID NO:3, an analog of a peptide of SEQ ID NO:3, a fragment of SEQ ID NO:3, or an analog of a fragment of SEQ ID NO:3. In specific embodiments, the analog of SEQ ID NO:3 is a peptide that is a conservative variant of SEQ ID NO:3. The method may further comprise administering a second composition that stimulates FSH production in the mammal. The second composition that stimulates FSH production comprises an anti-estrogenic compound, e.g., anti-estrogenic compound is selected from the group consisting of clomiphene and letrazole.

Also described herein is a method of stimulating LH production in a mammal comprising administering to the mammal a composition comprising a Kiss-1 derived protein.

The methods of the present invention also encompass methods for the therapeutic intervention of infertility in a mammal comprising suppressing endogenous gonadotropins production in the mammal, administering to the mammal a composition Kiss-1 related peptide in an amount effect to stimulate ovarian follicle growth in the mammal; and inducing ovulation in the mammal to produce an ovum. The infertility treatment methods may further comprise administering a gonadotropin preparation to stimulate ovarian follicle growth. Such a gonadotropin preparation may comprise urinary or recombinant FSH or HMG, with or without recombinant LH. In particular embodiments, the gonadotropin preparation comprises recombinant FSH. In specific embodiments, the induction of ovulation comprises administering to the mammal a composition comprising HCG, native LHRH, LHRH agonists or recombinant LH. In more specific embodiments, the suppression of endogenous gonadotropin production in the mammal comprises administering a GnRH antagonist.

In preferred embodiments, the Kiss-1 derived peptide is administered in combination with an anti-estrogenic agent in a combined amount effective to stimulate FSH production in the mammal. In other aspects of the invention, the methods described herein may be used in a mammal that is non-responsive to standard gonadotropin stimulation of normal ovulation. In specific embodiments, the infertility disorder is hypogonadotropic hypogonadism. Other methods involve treatment of the infertility disorder polycystic ovary syndrome.

The infertility treatment methods described herein further comprise intrauterine insemination of the ovum by sperm injection. In other embodiments, the methods further comprise harvesting oocytes 12 days after the initial stimulation of the ovarian follicle production. In more specific embodiments the methods further comprise fertilizing the harvested oocytes in vitro, and culturing the harvested, fertilized oocytes to the 4-8 cell stage. In preferred embodiments, the methods further comprise transferring the 4-8 cell stage fertilized oocytes to the uterus of a mammal. In specific embodiments, the fertilized oocytes are transferred to the uterus of the same mammal from which the oocytes were harvested. However, in other embodiments, the fertilized oocytes are transferred to the uterus of a different mammal from which the oocytes were harvested.

The methods of the invention also alleviate one or more of the symptoms hypogonadotropic hypogonadism in a subject by administering to the individual a therapeutically effective amount of a Kiss-1 derived peptide. In such embodiments, the subject is a male subject. The subject suffering from hypogonadotropic hypogonadism may be a female subject.

The invention further contemplates a composition comprising a Kiss-1 derived protein for use in the treating of infertility. In yet further embodiments, the invention provides a kit for the treatment of infertility, wherein the kit comprises a first composition comprising a Kiss-1 derived protein in a pharmaceutically acceptable formulation, and a second composition for the treatment of infertility. Preferably, the second composition for the treatment of infertility comprises an FSH preparation in a pharmaceutically acceptable formulation. In specific embodiments, the FSH preparation is selected from the group consisting of a urinary FSH preparation and a recombinant FSH. Preferably, the FSH is human FSH. In specific embodiments, the FSH is present in a unit dose of between about 5 IU FSH and about 75 IU FSH. The kits also may further comprise a third composition comprising LH in a pharmaceutically acceptable formulation. More particularly, the LH is present in a unit dose of between about 75 IU LH and about 150 IU LH.

Other features and advantages of the invention will become apparent from the following detailed description. It should be understood, however, that the detailed description and the specific examples, while indicating preferred embodiments of the invention, are given by way of illustration only, because various changes and modifications within the spirit and scope of the invention will become apparent to those skilled in the art from this detailed description.

### BRIEF DESCRIPTION OF THE FIGURES

The following drawings form part of the present specification and are included to further illustrate aspects of the present invention. The invention may be better understood by reference to the drawings in combination with the detailed description of the specific embodiments presented herein.

FIGS. 1A-P shows a comparison of the in vivo effects of GnRH and Kiss1 on the release of LH (FIGS. 1A-H) and FSH (FIGS. 1I-P) over a 45 minute time period. FIGS. 1A and 1B shows the effect of vehicle alone on LH release. FIGS. 1C and 1D shows the effect of 90 ng/kg GnRH-I administration on LH release. FIGS. 1E and 1F shows the effect of 9 μg/kg GnRH-I administration on LH release. FIGS. 1G and 1H shows the effect of 4 μg/kg Kiss-I administration on LH release. FIG. 1I and 1J shows the effect of vehicle alone on FSH release. FIGS. 1K and 1L shows the effect of 90 ng/kg GnRH-I administration on FSH release. FIGS. 1M and 1N shows the effect of 9 μg/kg GnRH-I administration on FSH release. FIGS. 1O and 1P shows the effect of 4 μg/kg Kiss-I administration on FSH release.

FIG. 2A-2B. Effects of Kiss-1 by i.v. injection on plasma LH (FIG. 2A) and FSH (FIG. 2B) levels, and prevention of these effects by pretreatment of GnRH antagonist, Antide, in estrogen-treated ovariectomized rats. Exclusive letters indicate statistical difference at P<0.05 determined by one-way ANOVA followed by Fisher's Least Significant Difference (Fisher's LSD) analysis as a post-hoc test.

FIG. 3A-3B. Effects of Kiss-1 by i.c.v. injection on plasma LH (FIG. 3A) and FSH (FIG. 3B) levels in estrogen-treated ovariectomized rats. Exclusive letters indicate statistical difference at P<0.05 determined by one-way ANOVA followed by Fisher's LSD as a post-hoc test.

### DESCRIPTION OF THE PREFERRED EMBODIMENTS

Infertility has been treated through a variety of mechanisms, most of which rely on stimulating or augmenting the effects of FSH and/or LH. Prior to the present invention, the first course of intervention in combating infertility has been to employ anti-estrogen compounds such as clomiphene or letrazole. However, it has been discovered that these agents have undesirable side effects. Thus there remains a need for effective additional treatments for infertility. The present invention provides novel methods and compositions for the stimulation of endogenous gonadotropin production. More particularly, the methods and compositions of the present invention produce a stimulation of FSH and/or LH production and/or release. This stimulation is advantageous in that it can be exploited in the treatment of various fertility disorders.

The present invention describes methods of treating infertility by administering compositions comprising Kiss-1-peptide related proteins. Methods and compositions for achieving this beneficial outcome are described in further detail herein below.

Kiss-1 Protein and its Receptor

The present invention is directed to methods of treating a variety of infertility disorders by administering Kiss-1 related proteins. In preferred aspects of the invention it has been found that Kiss-1 and peptides derived therefrom may be used to regulate the hypothalamic-pituitary-gonadal (H-P-G) axis and to modulate the production and circulating concentration of various hormones, including luteinizing releasing hormone (LHRH) (also known as GnRH), FSH, LH, as well as gonadal steroids such as estrogen and testosterone. In specific examples, it has been demonstrated that a Kiss-1 protein derived agonist of GPCR54, stimulates the production and release of FSH and/or LH. For example, it has been shown by the inventors that a single administration of the 10 amino acid RFamide of SEQ ID NO:3 was sufficient to drive LH and FSH production similar to GnRH. This finding importantly reveals that the level of induction of FSH production is in a therapeutically relevant range. As the H-P-G axis is involved in various aspects of the reproductive system, including but not limited to, timing of puberty, maintenance of fertility and in the time of onset of menopause, the stimulants of the invention may be used in the treatment of a wide variety of fertility disorders. The following section is directed to a description of Kiss-1 protein and compounds that may be derived therefrom that may be useful in the treatment of infertility.

G protein-coupled receptors (GPCRs) are a large family of membrane receptors. Hundreds of genes that belong to this family have been identified but in many instances their ligands and functions remain unelucidated. GPCR54 is one such orphan GPCR that was originally cloned from rat brain and found to be widely expressed in the rat central nervous system, including the hypothalamus, midbrain, pons, medulla, hippocampus, and amygdale. The gene encoding human GPCR54 is present in a bacterial artificial chromosome mapped to chromosome 2 (GenBank accession number AC023583). Recently, it has been shown that a placental tissue extract contains active peptides derived from metastasis suppressor gene KiSS-1 (SEQ ID NO:1; see also GenBank accession no. NM_{—}002256 and SwissProt accession no. Q15726) that are agonists of GPCR54 (Kotani et al., J. Biol. Chem., 276(37):34631-34636, 2001). The peptides (termed kisspeptins) described in Kotani were 54-, 14-, and 13-amino acids in length (SEQ ID NO: 4, SEQ ID NO:5 and SEQ ID NO:6, respectively) and contained a RF-amide at the C-terminus. These peptides are derived from the full-length Kiss-1 protein (SEQ ID NO:2). The present invention for the first time describes that compositions comprising Kiss-1 and kisspeptins may be used in the treatment of infertility and for the augmentation of gonadotropin (especially FSH and/or LH) production and/or release.

As used herein the term “Kiss-1 derived protein” is intended to encompass any protein that is derived from the sequence of SEQ ID NO:2, is a fragment of SEQ ID NO:2, or an analog or conservative variant of a protein of SEQ ID NO:2 that has any agonist effect on a GPCR54 receptor or a variant of a GPCR54 receptor. In the present invention a Kiss-1 derived peptide having the sequence of YNWNSFGLRF (SEQ ID NO:3) is a particularly preferred kisspeptin that may be used in the treatment of infertility. However, it should be understood that any variant, analog or fragment of SEQ ID NO:2 may be used in the methods of the present invention. Indeed, it has been found that RFamide peptides derived from Kiss-1 protein as short as 5 amino acids in length possess activity as agonists of GPCR54. Thus, it is contemplated that sequences such as FGLRF (SEQ ID NO:7); SFGLRF (SEQ ID NO:8); NSFGLRF (SEQ ID NO:9); WNSFGLRF (SEQ ID NO:10); NWNSFGLRF (SEQ ID NO:11) or variants or analogs thereof that possess receptor agonist activity will be useful in the present invention.

Other specific kisspeptins that may be useful herein include but are not limited to a fragment of Kiss-1 having amino acids 93-121 of SEQ ID NO:1, wherein the C-terminus of the fragment is RFamide derived from the RF at positions 120-121 of SEQ ID NO:1; a fragment of Kiss-1 having amino acids 106-121 of SEQ ID NO:1, wherein the C-terminus of the fragment is RFamide derived from the RF at positions 120-121 of SEQ ID NO:1, wherein the C-terminus of the fragment is RFamide derived from the RF at positions 120-121 of SEQ ID NO:1, a fragment of Kiss-1 having amino acids 108-121 of SEQ ID NO:1, wherein the C-terminus of the fragment is RFamide derived from the RF at positions 120-121 of SEQ ID NO:1, a fragment of Kiss-1 having amino acids 68-121 of SEQ ID NO:1, wherein the C-terminus of the fragment is RFamide derived from the RF at positions 120-121 of SEQ ID NO:1; a fragment of Kiss-1 having amino acids 94-121 of SEQ ID NO:1, wherein the C-terminus of the fragment is RFamide derived from the RF at positions 120-121 of SEQ ID NO:1; a fragment of Kiss-1 having amino acids 107-121 of SEQ ID NO:1, wherein the C-terminus of the fragment is RFamide derived from the RF at positions 120-121 of SEQ ID NO:1; a fragment of Kiss-1 having amino acids 109-121 of SEQ ID NO:1, wherein the C-terminus of the fragment is RFamide derived from the RF at positions 120-121 of SEQ ID NO:1; a fragment of Kiss-1 having amino acids 112-121 of SEQ ID NO:1, wherein the C-terminus of the fragment is RFamide derived from the RF at positions 120-121 of SEQ ID NO:1; and a fragment of Kiss-1 having amino acids 114-121 of SEQ ID NO:1, wherein the C-terminus of the fragment is RFamide derived from the RF at positions 120-121 of SEQ ID NO:1.

In preferred embodiments, the peptide may comprise amino acids 110-121 of SEQ ID NO:1; amino acids 109-121 of SEQ ID NO:1, 108-121 of SEQ ID NO:1; 107-121 of SEQ ID NO:1; 106-121 of SEQ ID NO:1; 105-121 of SEQ ID NO:1; 104-121 of SEQ ID NO:1; 103-121 of SEQ ID NO:1; 102-121 of SEQ ID NO:1; 101-121 of SEQ ID NO:1; 100-121 of SEQ ID NO:1; 99-121 of SEQ ID NO:1; 98-121 of SEQ ID NO:1; 97-121 of SEQ ID NO:1; 96-121 of SEQ ID NO:1; 95-121 of SEQ ID NO:1; 94-121 of SEQ ID NO:1; 93-121 of SEQ ID NO:1; 92-121 of SEQ ID NO:1; 91-121 of SEQ ID NO:1; 90-121 of SEQ ID NO:1; 89-121 of SEQ ID NO:1; 88-121 of SEQ ID NO:1; 87-121 of SEQ ID NO:1; 86-121 of SEQ ID NO:1; 85-121 of SEQ ID NO:1; 84-121 of SEQ ID NO:1; 83-121 of SEQ ID NO:1; 82-121 of SEQ ID NO:1; 81-121 of SEQ ID NO:1; 80-121 of SEQ ID NO:1; 79-121 of SEQ ID NO:1; 78-121 of SEQ ID NO:1; 77-121 of SEQ ID NO:1; 76-121 of SEQ ID NO:1; 75-121 of SEQ ID NO:1; 74-121 of SEQ ID NO:1; 73-121 of SEQ ID NO:1; 72-121 of SEQ ID NO:1; 71-121 of SEQ ID NO:1; 70-121 of SEQ ID NO:1; 69-121 of SEQ ID NO:1; 68-121 of SEQ ID NO:1; 67-121 of SEQ ID NO:1; 66-121 of SEQ ID NO:1; 65-121 of SEQ ID NO:1; 64-121 of SEQ ID NO:1; 63-121 of SEQ ID NO:1; 62-121 of SEQ ID NO:1; 61-121 of SEQ ID NO:1; 60-121 of SEQ ID NO:1; 59-121 of SEQ ID NO:1; 58-121 of SEQ ID NO:1; 57-121 of SEQ ID NO:1; 56-121 of SEQ ID NO:1; 55-121 of SEQ ID NO:1; 54-121 of SEQ ID NO:1; 53-121 of SEQ ID NO:1; 52-121 of SEQ ID NO:1; 51-121 of SEQ ID NO:1; 50-121 of SEQ ID NO:1; 49-121 of SEQ ID NO:1; 48-121 of SEQ ID NO:1; 47-121 of SEQ ID NO:1; 46-121 of SEQ ID NO:1; 45-121 of SEQ ID NO:1; 44-121 of SEQ ID NO:1; 43-121 of SEQ ID NO:1; 42-121 of SEQ ID NO:1; 41-121 of SEQ ID NO:1; 40-121 of SEQ ID NO:1; 39-121 of SEQ ID NO:1; 38-121 of SEQ ID NO:1; 37-121 of SEQ ID NO:1; 36-121 of SEQ ID NO:1; 35-121 of SEQ ID NO:1; 34-121 of SEQ ID NO:1; 33-121 of SEQ ID NO:1; 32-121 of SEQ ID NO:1; 31-121 of SEQ ID NO:1; 30-121 of SEQ ID NO:1; 29-121 of SEQ ID NO:1; 28-121 of SEQ ID NO:1; 27-121 of SEQ ID NO:1; 26-121 of SEQ ID NO:1; 25-121 of SEQ ID NO:1; 24-121 of SEQ ID NO:1; 23-121 of SEQ ID NO:1; 22-121 of SEQ ID NO:1; 21-121 of SEQ ID NO:1; 20-121 of SEQ ID NO:1; 19-121 of SEQ ID NO:1; 18-121 of SEQ ID NO:1; 17-121 of SEQ ID NO:1; 16-121 of SEQ ID NO:1; 15-121 of SEQ ID NO:1; 14-121 of SEQ ID NO:1; 13-121 of SEQ ID NO:1; 12-121 of SEQ ID NO:1; 11-121 of SEQ ID NO:1; 10-121 of SEQ ID NO:1; 9-121 of SEQ ID NO:1; 8-121 of SEQ ID NO:1; 7-121 of SEQ ID NO:1; 6-121 of SEQ ID NO:1; 5-121 of SEQ ID NO:1; 4-121 of SEQ ID NO:1; 3-121 of SEQ ID NO:1; 2-121 of SEQ ID NO:1; and 1-121 of SEQ ID NO:1.

A particularly preferred peptide of the present invention is a peptide having the sequence of SEQ ID NO:3. However, it is also contemplated that conservative substitution of amino acid residues of this peptide also may be produced that nonetheless retain the three-dimensional conformation structure of the peptide of SEQ ID NO:3 and/or retain the functional activity of the peptide of SEQ ID NO:3 as an agonist of a GPCR54 receptor and/or stimulant of FSH and/or LH production. The term “conservative substitution” as used herein denotes the replacement of an amino acid residue by another, biologically similar residue with respect to hydrophobicity, hydrophilicity, cationic charge, anionic charge, shape, polarity, conformational tendency, and the like. Examples of conservative substitutions include the substitution of one hydrophobic residue such as isoleucine, valine, leucine, alanine, cysteine, glycine, phenylalanine, proline, tryptophan, tyrosine, norleucine or methionine for another, or the substitution of one polar residue for another, such as the substitution of arginine for lysine, glutamic acid for aspartic acid, or glutamine for asparagine, and the like. Neutral hydrophilic amino acids which can be substituted for one another include asparagine, glutamine, serine and threonine. The term “conservative substitution” also includes the use of a substituted or modified amino acid in place of an unsubstituted parent amino acid provided that antibodies raised to the substituted polypeptide also immunoreact with the unsubstituted polypeptide. By “substituted” or “modified” the present invention includes those amino acids that have been altered or modified from naturally occurring amino acids.

As such, it should be understood that in the context of the present invention, a conservative substitution is recognized in the art as a substitution of one amino acid for another amino acid that has similar properties. Exemplary conservative substitutions are set out in e.g., Alternatively, conservative amino acids can be grouped as described in Lehninger, [Biochemistry, Second Edition; Worth Publishers, Inc. NY:NY (1975), pp. 71-77]. Those of skill in the art are aware of numerous tables that indicate specific conservative substitutions. One exemplary such table is provided below:

Table of Exemplary Conservative Substitutions

| Original | |
|---|---|
| Residue | Exemplary Substitution |
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe, |
| Leu (L) | Ile, Val, Met, Ala, Phe |
| Lys (K) | Arg, Gln, Asn |
| Met (M) | Leu, Phe, Ile |
| Phe (F) | Leu, Val, Ile, Ala |
| Pro (P) | Gly |
| Ser (S) | Thr |
| Thr (T) | Ser |
| Trp (W) | Tyr |
| Tyr (Y) | Trp, Phe, Thr, Ser |
| Val (V) | Ile, Leu, Met, Phe, Ala |

Any conservative variant of YNWNSFGLRF (SEQ ID NO:3) is contemplated to be a useful peptide of the present invention as long as such a variant retains its property of being an agonist of a GPCR54 and/or the ability to stimulate FSH and/or LH production. Such activities may be readily assessed as described herein below.

Other novel peptides include mutants of any of the sequences discussed herein above that has similar conformational tendencies. The similarity of conformational tendencies of amino acids can be obtained by comparing their Ramachandran plots, as shown in the following table:

TABLE
Similarity of amino acids in terms of conformational tendencies

| | | | |
|---|---|---|---|
| | Highly similar | Medialy similar | Lowly similar |
| Original | residues | residues | residues |

| | | | |
|---|---|---|---|
| ALA | GLU ARG GLN HIS LEU PHE LYS | TYR SER CYS MET ASN THR | ASP TRP ILE VAL PRO GLY |
| ARG | LYS HIS GLN PHE GLU LEU TYR | TRP ALA CYS MET THR SER | VAL ILE ASN ASP PRO GLY |
| ASN | HIS ASP PHE TYR SER GLN ARG | CYS ALA GLU LEU LYS MET | TRP THR ILE VAL PRO GLY |
| ASP | ASN HIS ARG ALA PHE SER GLU | GLN LYS TYR LEU CYS MET | THR TRP VAL ILE PRO GLY |
| CYS | TRP GLN MET HIS TYR PHE ARG | LEU GLU VAL LYS ILE ALA | ASN THR SER ASP PRO GLY |
| GLN | PHE HIS ARG GLU LYS CYS LEU | MET TYR TRP VAL ILE ALA | THR ASN SER ASP PRO GLY |
| GLU | GLN ARG LYS LEU PHE ALA TYR | HIS TRP THR VAL CYS MET | ILE SER ASN ASP PRO GLY |
| GLY | ALA SER ASP GLU ASN LYS THR | ARG PHE LEU HIS TYR GLN | CYS MET TRP VAL ILE PRO |
| HIS | PHE GLN ARG TYR CYS LYS LEU | ASN MET GLU TRP ALA THR | ILE VAL SER ASP PRO GLY |
| ILE | VAL MET TRP LEU GLN PHE GLU | TYR LYS THR HIS ARG CYS | ALA ASN SER ASP PRO GLY |
| LEU | LYS ARG PHE GLN GLU HIS ILE | TYR MET TRP VAL ALA THR | CYS ASN SER ASP PRO GLY |
| LYS | ARG LEU GLN GLU PHE TYR HIS | ALA MET THR ILE TRP CYS | VAL ASN SER ASP PRO GLY |
| MET | TRP VAL GLN CYS ILE PHE LEU | HIS ARG TYR LYS GLU THR | ALA ASN SER ASP PRO GLY |
| PHE | HIS GLN TYR ARG LEU LYS TRP | MET GLU CYS VAL ASN ILE | ALA THR SER ASP PRO GLY |
| PRO | ILE VAL MET TRP LEU GLN CYS | ARG THR GLU LYS ALA HIS | PHE TYR ASN SER ASP GLY |
| SER | ALA ASN ARG HIS GLU TYR PHE | GLN THR LEU LYS ASP CYS | MET TRP VAL ILE PRO GLY |
| THR | VAL LEU TYR GLU ARG LYS GLN | ILE HIS PHE MET TRP ALA | CYS SER ASN ASP PRO GLY |
| TRP | MET CYS GLN ILE PHE ARG VAL | TYR LEU GLU HIS LYS THR | ALA ASN SER ASP PRO GLY |
| TYR | PHE HIS GLN LYS ARG CYS LEU | GLU TRP ASN MET ALA THR | ILE VAL SER ASP PRO GLY |
| VAL | ILE MET TRP GLN PHE LEU THR | GLU CYS ARG TYR LYS HIS | ALA SER ASN ASP PRO GLY |

In addition to the basic amino acid structure of the peptides, it is contemplated that the Kiss-1 derived proteins/peptides may be modified to enhance their uptake, circulation, and/or other modifications to render the peptides more therapeutically effective. For example, it has been discovered herein that kisspeptins and Kiss-1 derived proteins have a beneficial effect on stimulating fertility related hormones by acting on sites within the central nervous system, and likely within the blood brain barrier. Therefore, any modification that allows the targeting/uptake of the peptides/protein to a site within the H-P-G axis will be useful.

For example, it may be desirable to prevent the degradation of the peptides in order to prolong the effects thereof. This may be achieved through the use of non-hydrolyzable peptide bonds, which are known in the art, along with procedures for synthesis of peptides containing such bonds. Non-hydrolyzable bonds include —[CH₂NH]— reduced amide peptide bonds, —[COCH₂]— ketomethylene peptide bonds, —[CH(CN)NH]— (cyanomethylene)amino peptide bonds, —[CH₂ CH(OH)]— hydroxyethylene peptide bonds, —[CH₂O]— peptide bonds, and —[CH₂S]—thiomethylene peptide bonds (see e.g., U.S. Pat. No. 6,172,043), and peptoids. Peptoids are N-substituted glycines that can be used to optimize uptake of a drug agent of interest. Peptoids have been used to create cationic lipid-like compounds (Murphy et al., Proc. Natl. Acad. Sci. 95:1517, 1998) and can be synthesized using standard methods (e.g., Zuckermann et al. J. Am. Chem. Soc 114:10646, 1992; Zuckermann et al. lit. J. Peptide Protein Res. 40:497, 1992). Combinations of cationic lipids and peptoids, liptoids, can also be used to optimize uptake of the subject compositions (Hunag et al. Chemistry and Biology. 5:345, 1998). Liptoids can be synthesized by elaborating peptoids and coupling the amino terminal submonomer to a lipid via its amino group (Hunag et al. Chemistry and Biology, 5:345, 1998). Those of skill in the art are referred to e.g., U.S. Patent Publication No. 20030187188 (incorporated herein by reference), which describes the synthesis of peptoid substituted compounds. Methods of making peptoid containing serine protease inhibitors are described in U.S. Pat. No. 6,548,638; U.S. Patent Publication No. 20030176642 (incorporated herein by reference).

Kiss-1 derived proteins useful in the invention can be linear, or maybe circular or cyclized by natural or synthetic means. For example, disulfide bonds between cysteine residues may cyclize a peptide sequence. Bifunctional reagents can be used to provide a linkage between two or more amino acids of a peptide. Other methods for cyclization of peptides, such as those described by Anwer et al. (Int. J Pep. Protein Res. 36:392-399, 1990) and Rivera-Baeza et al. (Neuropeptides 30:327-333, 1996) are also known in the art.

Rational drug design may be used to produce structural analogs of the presently known biologically active Kiss-1 derived polypeptides. By creating such analogs, it is possible to fashion Kiss-1 derived proteins which are more active or stable than the natural molecules which have different susceptibility to alteration or which may affect the function of various other molecules. In one approach, one would generate a three-dimensional structure for Kiss-1 derived protein of interest or a fragment thereof e.g., this can be accomplished by x-ray crystallography, computer modeling or by a combination of both approaches. An alternative approach, “alanine scan,” involves the random replacement of residues throughout molecule with alanine, and the resulting affect on function determined.

Multiple sequence alignment of Kiss-1 and its sequence analogs such as those from different species or those of related ligand family provide further guidance of rational substitutions. The residues in the Kiss-1 sequence may be substituted by the corresponding residues from the related sequence from a different species or related ligand family.

It also is possible to isolate a Kiss-1-specific antibody, selected by a functional assay, and then solve its crystal structure. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated by producing antibodies specific for Kiss-1 and then using such an antibody as the antigen.

Thus, one may design drugs which have improved Kiss-1 derived protein activity or which act as stimulators, agonists, of Kiss-1 protein or the GPCR54 receptor. By virtue of the availability of cloned Kiss-1 sequences, sufficient amounts of various Kiss-1 derived proteins can be produced to perform crystallographic studies. In addition, knowledge of the polypeptide sequences permits computer employed predictions of structure-function relationships.

Furthermore, nonpeptide analogs of Kiss-1 derived proteins which provide a stabilized structure or lessened biodegradation, are also contemplated. Peptide mimetic analogs can be prepared based on a Kiss-1 derived protein by replacing one or more amino acid residues of the protein of interest by nonpeptide moieties. Preferably, the nonpeptide moieties permit the peptide to retain its natural confirmation, or stabilize a preferred, e.g., bioactive, confirmation. One example of methods for preparation of nonpeptide mimetic analogs from peptides is described in Nachman et al., Regul. Pept. 57:359-370 (1995). Peptide as used herein embraces all of the foregoing.

The Kiss-1 derived proteins used in the therapeutic methods of the present invention may be modified in order to improve their therapeutic efficacy. Such modification of therapeutic compounds may be used to decrease toxicity, increase circulatory time, or modify biodistribution. For example, the toxicity of potentially important therapeutic compounds can be decreased significantly by combination with a variety of drug carrier vehicles that modify biodistribution. An elegant example is the recently approved Wyeth-Ayerst chemotherapeutic Mylotarg™, which is composed of the potent cytotoxic agent calicheamicin linked to an antibody that is targeted to CD33 (found on the surface of certain leukemic cells). This antibody-drug conjugate had a dramatically improved therapeutic index versus the free drug. The Kiss-1 derived proteins have their therapeutic effect on the H-P-G axis by acting on the pituitary in order to stimulate FSH production and/or release. As such, any modification that allows the peptide to be taken up and have an effect along the H-P-G axis will be useful.

A strategy for improving drug viability is the utilization of water-soluble polymers. Various water-soluble polymers have been shown to modify biodistribution, improve the mode of cellular uptake, change the permeability through physiological barriers, and modify the rate of clearance from the body. (Greenwald et al., Crit Rev Therap Drug Carrier Syst. 2000; 17:101-161; Kopecek et al., J Controlled Release., 74:147-158, 2001). To achieve either a targeting or sustained-release effect, water-soluble polymers have been synthesized that contain drug moieties as terminal groups, as part of the backbone, or as pendent groups on the polymer chain.

Polyethylene glycol (PEG), has been widely used as a drug carrier, given its high degree of biocompatibility and ease of modification. Harris et al., Clin Pharmacokinet. 2001; 40(7):539-51 Attachment to various drugs, proteins, and liposomes has been shown to improve residence time and decrease toxicity. (Greenwald et al., Crit Rev Therap Drug Carrier Syst. 2000; 17:101-161; Zalipsky et al., Bioconjug Chem. 1997; 8:111-118). PEG can be coupled to active agents through the hydroxyl groups at the ends of the chain and via other chemical methods; however, PEG itself is limited to at most two active agents per molecule. In a different approach, copolymers of PEG and amino acids were explored as novel biomaterials which would retain the biocompatibility properties of PEG, but which would have the added advantage of numerous attachment points per molecule (providing greater drug loading), and which could be synthetically designed to suit a variety of applications (Nathan et al., Macromolecules. 1992; 25:4476-4484; Nathan et al., Bioconj Chem. 1993; 4:54-62).

Those of skill in the art are aware of PEGylation techniques for the effective modification of drugs. For example, drug delivery polymers that consists of alternating polymers of PEG and tri-functional monomers such as lysine and cysteine have been used by VectraMed (Plainsboro, N.J.) or Shearwater (N.J.). The PEG chains (typically 2000 daltons or less) are linked to the a- and e-amino groups of lysine through stable urethane linkages. Such copolymers retain the desirable properties of PEG, while providing reactive pendent groups (the carboxylic acid groups of lysine) at strictly controlled and predetermined intervals along the polymer chain. The reactive pendent groups can be used for derivatization, cross-linking, or conjugation with other molecules. These polymers are useful in producing stable, long-circulating pro-drugs by varying the molecular weight of the polymer, the molecular weight of the PEG segments, and the cleavable linkage between the drug and the polymer. The molecular weight of the PEG segments affects the spacing of the drug/linking group complex and the amount of drug per molecular weight of conjugate (smaller PEG segments provides greater drug loading). In general, increasing the overall molecular weight of the block co-polymer conjugate will increase the circulatory half-life of the conjugate. Nevertheless, the conjugate must either be readily degradable or have a molecular weight below the threshold-limiting glomular filtration (e.g., less than 45 kDa). Thus, PEgylated Kiss-1 derived peptides of the invention should preferably be in the range of between 20 kDa and 35 kDa in molecular weight.

The effective modification of drugs is not limited to the PEGylation techniques. Glycosylation of the molecule, fusion to another molecule, such as antibodies or human serum albumin or the combinations are examples of other modifications that may be used to facilitate a more effective or efficient drug delivery.

In addition, to the polymer backbone being important in maintaining circulatory half-life, and biodistribution, linkers may be used to maintain the therapeutic agent in a pro-drug form until released from the backbone polymer by a specific trigger, typically enzyme activity in the targeted tissue. For example, this type of tissue activated drug delivery is particularly useful where delivery to a specific site of biodistribution is required and the therapeutic agent is released at or near the site of pathology. Linking group libraries for use in activated drug delivery are known to those of skill in the art and may be based on enzyme kinetics, prevalence of active enzyme, and cleavage specificity of the selected disease-specific enzymes (see e.g., technologies of established by VectraMed, Plainsboro, N.J.). Such linkers may be used in modifying the Kiss-1 derived proteins described herein for therapeutic delivery.

Methods of Making Kiss-1 Derived Peptides

The present invention provides proteins and peptides for use in medicaments for the treatment of infertility. Such proteins or peptides may be produced by conventional automated peptide synthesis methods or by recombinant expression. General principles for designing and making proteins are well known to those of skill in the art.

A. Automated Solid-Phase Peptide Synthesis

The peptides or indeed even the full length Kiss-1 of the invention can be synthesized in solution or on a solid support in accordance with conventional techniques. Various automatic synthesizers are commercially available and can be used in accordance with known protocols. See, for example, Stewart and Young, Solid Phase Peptide Synthesis, 2d. ed., Pierce Chemical Co., (1984); Tam et al., J. Am. Chem. Soc., 105:6442, (1983); Merrifield, Science, 232: 341-347, (1986); and Barany and Merrifield, The Peptides, Gross and Meienhofer, eds, Academic Press, New York, 1-284, (1979), each incorporated herein by reference. The novel Kiss-1 derived proteins of the invention can be readily synthesized and then screened in GPCR54 receptor binding/activity assays.

For example, the peptides may be synthesized by solid-phase technology employing an exemplary peptide synthesizer such as a Model 433A from Applied Biosystems Inc. The purity of any given peptide substrate, generated through automated peptide synthesis or through recombinant methods may be determined using reverse phase HPLC analysis. Chemical authenticity of each peptide may be established by any method well known to those of skill in the art. In preferred embodiments, the authenticity may be established by mass spectrometry. Additionally, the peptides may be quantitated using amino acid analysis in which microwave hydrolyses are conducted. Such analyses may use a microwave oven such as the CEM Corporation's MDS 2000 microwave oven. The peptide (approximately 2 μg protein) is contacted with e.g., 6 N HCl (Pierce Constant Boiling e.g., about 4 ml) with approximately 0.5% (volume to volume) phenol (Mallinckrodt). Prior to the hydrolysis, the samples are alternately evacuated and flushed with N₂. The protein hydrolysis is conducted using a two-stage process. During the first stage, the peptides are subjected to a reaction temperature of about 100° C. and held that temperature for 1 minute. Immediately after this step, the temperature is increased to 150° C. and held at that temperature for about 25 minutes. After cooling, the samples are dried and amino acid from the hydrolysed peptides samples are derivatized using 6-aminoquinolyl-N-hydroxysuccinimidyl carbamate to yield stable ureas that fluoresce at 395 nm (Waters AccQ Tag Chemistry Package). The samples may be analyzed by reverse phase HPLC and quantification may be achieved using an enhanced integrator.

B. Recombinant Protein Production.

As an alternative to automated peptide synthesis, recombinant DNA technology may be employed wherein a nucleotide sequence which encodes a peptide of the invention is inserted into an expression vector, transformed or transfected into an appropriate host cell and cultivated under conditions suitable for expression as described herein below. Recombinant methods are especially preferred for producing longer polypeptides that comprise peptide sequences of the invention.

A variety of expression vector/host systems may be utilized to contain and express the peptide or protein coding sequence. These include but are not limited to microorganisms such as bacteria transformed with recombinant bacteriophage, plasmid or cosmid DNA expression vectors; yeast transformed with yeast expression vectors; insect cell systems infected with virus expression vectors (e.g., baculovirus); plant cell systems transfected with virus expression vectors (e.g., cauliflower mosaic virus, CaMV; tobacco mosaic virus, TMV) or transformed with bacterial expression vectors (e.g., Ti or pBR322 plasmid); or animal cell systems. Mammalian cells that are useful in recombinant protein productions include but are not limited to VERO cells, HeLa cells, Chinese hamster ovary (CHO) cell lines, COS cells (such as COS-7), W138, BHK, HepG2, 3T3, RIN, MDCK, A549, PC12, K562 and 293 cells. Exemplary protocols for the recombinant expression of the peptide substrates or fusion polypeptides in bacteria, yeast and other invertebrates are known to those of skill in the art and a briefly described herein below.

Expression vectors for use in prokaryotic hosts generally comprise one or more phenotypic selectable marker genes. Such genes generally encode, e.g., a protein that confers antibiotic resistance or that supplies an auxotrophic requirement. A wide variety of such vectors are readily available from commercial sources. Examples include pSPORT vectors, pGEM vectors (Promega), pPROEX vectors (LTI, Bethesda, Md.), Bluescript vectors (Stratagene), pET vectors (Novagen) and pQE vectors (Qiagen). The DNA sequence encoding the given peptide substrate or fusion polypeptide is amplified by PCR and cloned into such a vector, for example, pGEX-3X (Pharmacia, Piscataway, N.J.) designed to produce a fusion protein comprising glutathione-S-transferase (GST), encoded by the vector, and a protein encoded by a DNA fragment inserted into the vector's cloning site. The primers for the PCR may be generated to include for example, an appropriate cleavage site. Treatment of the recombinant fusion protein with thrombin or factor Xa (Pharmacia, Piscataway, N.J.) is expected to cleave the fusion protein, releasing the substrate or substrate containing polypeptide from the GST portion. The pGEX-3×/Kiss-1 peptide construct is transformed into E. coli XL-1 Blue cells (Stratagene, La Jolla Calif.), and individual transformants were isolated and grown. Plasmid DNA from individual transformants is purified and partially sequenced using an automated sequencer to confirm the presence of the desired peptide or polypeptide encoding nucleic acid insert in the proper orientation. If the GST/Kiss-1 derived protein fusion protein is produced in bacteria as a soluble protein, it may be purified using the GST Purification Module (Pharmacia Biotech).

Alternatively, the DNA sequence encoding the predicted substrate containing fusion polypeptide may be cloned into a plasmid containing a desired promoter and, optionally, a leader sequence (see, e.g., Better et al., Science, 240:1041-43, 1988). The sequence of this construct may be confirmed by automated sequencing. The plasmid is then transformed into E. coli using standard procedures employing CaCl₂ incubation and heat shock treatment of the bacteria (Sambrook et al., supra). The transformed bacteria are grown in LB medium supplemented with carbenicillin, and production of the expressed protein is induced by growth in a suitable medium. If present, the leader sequence will effect secretion of the mature Kiss-1 peptide or fusion protein and be cleaved during secretion.

The secreted recombinant protein is purified from the bacterial culture media by the method described herein throughout. Similar systems for the recombinant protein in yeast host cells are readily commercially available, e.g., the Pichia Expression System (Invitrogen, San Diego, Calif.), following the manufacturer's instructions. Another alternative recombinant production may be achieved using an insect system. Insect systems for protein expression are well known to those of skill in the art. In one such system, Autographa californica nuclear polyhedrosis virus (AcNPV) is used as a vector to express foreign genes in Spodoptera frugiperda cells or in Trichoplusia larvae. The Kiss-1 coding sequence is cloned into a nonessential region of the virus, such as the polyhedrin gene, and placed under control of the polyhedrin promoter. Successful insertion of Kiss-1 will render the polyhedrin gene inactive and produce recombinant virus lacking coat protein coat. The recombinant viruses are then used to infect S. frugiperda cells or Trichoplusia larvae in which the Kiss-1 is expressed (Smith et al., J Virol 46: 584, 1983; Engelhard E K et al., Proc Nat Acad Sci 91: 3224-7, 1994).

Mammalian host systems for the expression of recombinant proteins also are well known to those of skill in the art. Host cell strains may be chosen for a particular ability to process the expressed protein or produce certain post-translation modifications that will be useful in providing protein activity. Such modifications of the polypeptide include, but are not limited to, acetylation, carboxylation, glycosylation, phosphorylation, lipidation and acylation. Post-translational processing which cleaves a “prepro” form of the protein may also be important for correct insertion, folding and/or unction. Different host cells such as CHO, HeLa, MDCK, 293, WI38, and the like have specific cellular machinery and characteristic mechanisms for such post-translational activities and may be chosen to ensure the correct modification and processing of the introduced, foreign protein.

It is preferable that the transformed cells are used for long-term, high-yield protein production and as such stable expression is desirable. Once such cells are transformed with vectors that contain selectable markers along with the desired expression cassette, the cells may be allowed to grow for 1-2 days in an enriched media before they are switched to selective media. The selectable marker is designed to confer resistance to selection and its presence allows growth and recovery of cells which successfully express the introduced sequences. Resistant clumps of stably transformed cells can be proliferated using tissue culture techniques appropriate to the cell.

A number of selection systems may be used to recover the cells that have been transformed for recombinant protein production. Such selection systems include, but are not limited to, HSV thymidine kinase, hypoxanthine-guanine phosphoribosyltransferase and adenine phosphoribosyltransferase genes, in tk−, hgprt− or aprt− cells, respectively. Also, anti-metabolite resistance can be used as the basis of selection for dhfr, which confers resistance to methotrexate; gpt, which confers resistance to mycophenolic acid; neo, which confers resistance to the aminoglycoside G418; als which confers resistance to chlorsulfuron; and hygro, which confers resistance to hygromycin. Additional selectable genes that may be useful include trpB, which allows cells to utilize indole in place of tryptophan, or hisD, which allows cells to utilize histinol in place of histidine. Markers that give a visual indication for identification of transformants include anthocyanins, b-glucuronidase and its substrate, GUS, and luciferase and its substrate, luciferin.

C. Expression Constructs for Recombinant Protein Production

In the recombinant production of the Kiss-1 derived proteins of the invention, it would be necessary to employ vectors comprising polynucleotide molecules for encoding the Kiss-1 derived proteins. Methods of preparing such vectors as well as producing host cells transformed with such vectors are well known to those skill in the art. The polynucleotide molecules used in such an endeavor may be joined to a vector, which generally includes a selectable marker and an origin of replication, for propagation in a host. These elements of the expression constructs are well known to those of skill in the art. Generally, the expression vectors include DNA encoding the given protein being operably linked to suitable transcriptional or translational regulatory sequences, such as those derived from a mammalian, microbial, viral, or insect gene. Examples of regulatory sequences include transcriptional promoters, operators, or enhancers, mRNA ribosomal binding sites, and appropriate sequences which control transcription and translation.

The terms “expression vector,” “expression construct” or “expression cassette” are used interchangeably throughout this specification and are meant to include any type of genetic construct containing a nucleic acid coding for a gene product in which part or all of the nucleic acid encoding sequence is capable of being transcribed.

The choice of a suitable expression vector for expression of the peptides or polypeptides of the invention will of course depend upon the specific host cell to be used, and is within the skill of the ordinary artisan. Methods for the construction of mammalian expression vectors are disclosed, for example, in Okayama and Berg (Mol. Cell. Biol. 3:280 (1983)); Cosman et al. (Mol. Immunol. 23:935 (1986)); Cosman et al. (Nature 312:768 (1984)); EP-A-0367566; and WO 91/18982.

The expression construct may further comprise a selectable marker that allows for the detection of the expression of a peptide or polypeptide. Usually the inclusion of a drug selection marker aids in cloning and in the selection of transformants, for example, neomycin, puromycin, hygromycin, DHFR, zeocin and histidinol. Alternatively, enzymes such as herpes simplex virus thymidine kinase (tk) (eukaryotic), b-galactosidase, luciferase, or chloramphenicol acetyltransferase (CAT) (prokaryotic) may be employed. Immunologic markers also can be employed. For example, epitope tags such as the FLAG system (IBI, New Haven, Conn.), HA and the 6×His system (Qiagen, Chatsworth, Calif.) may be employed. Additionally, glutathione S-transferase (GST) system (Pharmacia, Piscataway, N.J.), or the maltose binding protein system (NEB, Beverley, Mass.) also may be used. The selectable marker employed is not believed to be important, so long as it is capable of being expressed simultaneously with the nucleic acid encoding a gene product. Further examples of selectable markers are well known to one of skill in the art.

Expression requires that appropriate signals be provided in the vectors, such as enhancers/promoters from both viral and mammalian sources that may be used to drive expression of the nucleic acids of interest in host cells. Usually, the nucleic acid being expressed is under transcriptional control of a promoter. A “promoter” refers to a DNA sequence recognized by the synthetic machinery of the cell, or introduced synthetic machinery, required to initiate the specific transcription of a gene. Nucleotide sequences are operably linked when the regulatory sequence functionally relates to the DNA encoding the peptide substrate or the fusion polypeptide. Thus, a promoter nucleotide sequence is operably linked to a given DNA sequence if the promoter nucleotide sequence directs the transcription of the sequence. Similarly, the phrase “under transcriptional control” means that the promoter is in the correct location and orientation in relation to the nucleic acid to control RNA polymerase initiation and expression of the gene. Any promoter that will drive the expression of the nucleic acid may be used. The particular promoter employed to control the expression of a nucleic acid sequence of interest is not believed to be important, so long as it is capable of directing the expression of the nucleic acid in the targeted cell. Thus, where a human cell is targeted, it is preferable to position the nucleic acid coding region adjacent to and under the control of a promoter that is capable of being expressed in a human cell. Generally speaking, such a promoter might include either a human or viral promoter. Common promoters include, e.g., the human cytomegalovirus (CMV) immediate early gene promoter, the SV40 early promoter, the Rous sarcoma virus long terminal repeat, β-actin, rat insulin promoter, the phosphoglycerol kinase promoter and glyceraldehyde-3-phosphate dehydrogenase promoter, all of which are promoters well known and readily available to those of skill in the art, can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose. By employing a promoter with well-known properties, the level and pattern of expression of the protein of interest following transfection or transformation can be optimized. Inducible promoters also may be used.

Another regulatory element that is used in protein expression is an enhancer. These are genetic elements that increase transcription from a promoter located at a distant position on the same molecule of DNA. Where an expression construct employs a cDNA insert, one will typically desire to include a polyadenylation signal sequence to effect proper polyadenylation of the gene transcript. Any polyadenylation signal sequence recognized by cells of the selected transgenic animal species is suitable for the practice of the invention, such as human or bovine growth hormone and SV40 polyadenylation signals.

Also contemplated as an element of the expression cassette is a terminator. These elements can serve to enhance message levels and to minimize read through from the cassette into other sequences. The termination region which is employed primarily will be one selected for convenience, since termination regions for the applications such as those contemplated by the present invention appear to be relatively interchangeable. The termination region may be native with the transcriptional initiation, may be native to the DNA sequence of interest, or may be derived for another source.

D. Site-Specific Mutagenesis

Site-specific mutagenesis is another technique useful in the preparation of individual Kiss-1 derived proteins used in the methods of the invention. This technique employs specific mutagenesis of the underlying DNA (that encodes the amino acid sequence that is targeted for modification). The technique further provides a ready ability to prepare and test sequence variants, incorporating one or more of the foregoing considerations, by introducing one or more nucleotide sequence changes into the DNA. Site-specific mutagenesis allows the production of mutants through the use of specific oligonucleotide sequences that encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 25 nucleotides in length is preferred, with about 5 to 10 residues on both sides of the junction of the sequence being altered.

The technique typically employs a bacteriophage vector that exists in both a single stranded and double stranded form. Typical vectors useful in site-directed mutagenesis include vectors such as the M13 phage. These phage vectors are commercially available and their use is generally well known to those skilled in the art. Double stranded plasmids also are routinely employed in site directed mutagenesis, which eliminates the step of transferring the gene of interest from a phage to a plasmid.

In general, site-directed mutagenesis is performed by first obtaining a single-stranded vector, or melting of two strands of a double stranded vector which includes within its sequence a DNA sequence encoding the desired protein. An oligonucleotide primer bearing the desired mutated sequence is synthetically prepared. This primer is then annealed with the single-stranded DNA preparation, taking into account the degree of mismatch when selecting hybridization (annealing) conditions, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected that include recombinant vectors bearing the mutated sequence arrangement.

Of course, the above-described approach for site-directed mutagenesis is not the only method of generating potentially useful mutant peptide species and as such is not meant to be limiting. The present invention also contemplates other methods of achieving mutagenesis such as for example, treating the recombinant vectors carrying the gene of interest mutagenic agents, such as hydroxylamine, to obtain sequence variants. Other examples of generating potentially useful mutant peptides include yeast or phage display methods.

E. Protein Purification

It will be desirable to purify the peptides of the present invention. Protein purification techniques are well known to those of skill in the art. These techniques involve, at one level, the crude fractionation of the cellular milieu to polypeptide and non-polypeptide fractions. Having separated the peptides or polypeptides of the invention from other proteins, the polypeptides or peptides of interest may be further purified using chromatographic and electrophoretic techniques to achieve partial or complete purification (or purification to homogeneity).

Analytical methods particularly suited to the preparation of a pure peptide are ion-exchange chromatography, exclusion chromatography; polyacrylamide gel electrophoresis; isoelectric focusing. A particularly efficient method of purifying peptides is fast protein liquid chromatography (FPLC) or even high performance liquid chromatography (HPLC).

Certain aspects of the present invention concern the purification, and in particular embodiments, the substantial purification, of an encoded polypeptide, protein or peptide. The term “purified polypeptide, protein or peptide” as used herein, is intended to refer to a composition, isolated from other components, wherein the polypeptide, protein or peptide is purified to any degree relative to its naturally-obtainable state. A purified polypeptide, protein or peptide therefore also refers to a polypeptide, protein or peptide, free from the environment in which it may naturally occur.

Generally, “purified” will refer to a polypeptide, protein or peptide composition that has been subjected to fractionation to remove various other components, and which composition substantially retains its expressed biological activity. Where the term “substantially purified” is used, this designation will refer to a composition in which the polypeptide, protein or peptide forms the major component of the composition, such as constituting about 50%, about 60%, about 70%, about 80%, about 90%, about 95% or more of the proteins in the composition.

Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified polypeptide, protein or peptide.

Methods of Determining GPCR54 Receptor Agonist Activity of Kisspeptins

As indicated herein above, the Kiss-1 derived proteins of used herein are agonists of the GPCR54 receptor. This receptor is known to those of skill in the art and is encoded by the sequence set forth in e.g., GenBank accession no. AY0229541 (incorporated herein by reference). Methods of transfecting cells e.g. mammalian cells, with expression constructs that encode such a receptor expressed on the surface of the cell are well known in the art. (See, for example, U.S. Pat. Nos. 5,053,337; 5,155,218; 5,360,735; 5,472,866; 5,476,782; 5,516,653; 5,545,549; 5,556,753; 5,595,880; 5,602,024; 5,639,652; 5,652,113; 5,661,024; 5,766,879; 5,786,155; and 5,786,157, the disclosures of which are hereby incorporated by reference in their entireties into this application.)

Cells produced by such transfection may readily be used to test the Kiss-1 derived peptides described herein for binding activity to the orphan GPCR54 receptor, and to serve as agonists of this receptor. (See, for example, U.S. Pat. Nos. 5,053,337; 5,155,218; 5,360,735; 5,472,866; 5,476,782; 5,516,653; 5,545,549; 5,556,753; 5,595,880; 5,602,024; 5,639,652; 5,652,113; 5,661,024; 5,766,879; 5,786,155; and 5,786,157, the disclosures of which are hereby incorporated by reference in their entireties into this application.)

The term “agonist” is used throughout this application to indicate any Kiss-1 derived protein, peptide or peptide mimetic of a Kiss-1 derived protein compound which increases the activity of any of the GPCR54 receptor of the subject invention. Preferably, such an agent also stimulates the production and/or release of FSH and/or LH. Preferably, such a stimulation of release of one or other of these hormones is comparable to a GnRH-stimulated release of one or other of these hormones. The peptides also may be useful in stimulating GnRH release.

Typically, the receptor binding activity is determined by preparing a cell or a membrane preparation of a cell transfected with, and expressing a GPCR54 receptor, or obtaining a cell or a membrane preparation from a cell known to express said receptor, with the Kiss-1 derived protein to be tested under conditions permitting receptor-ligand binding. Exemplary such conditions are provided in e.g., Kotani et al., (J. Biol. Chem. 276(37):34661-34636, 2001). Following assays such as the receptor binding assays described in Kotani et al. will readily allow the determination of the receptor binding activity of the Kiss-1 derived protein being tested. Typically, such an activity may be compared to the binding of the kisspeptins known to bind the receptor.

Ligand Binding Assays

The binding properties of the Kiss-1 derived proteins may be tested using labeled ligand binding assays. For example, cells expressing the orphan receptor of this invention may be used to test the Kiss-1 proteins of for activity, for example, by labeled ligand binding assays. For example, the labeled kisspeptins such as those of SEQ ID NO:4, 5 or 6 may be labeled contacted with either membrane preparations or intact cells expressing the receptor in multi-well microtiter plates, together with unlabeled test Kiss-1 proteins to be tested, and binding buffer. Binding reaction mixtures are incubated for times and temperatures determined to be optimal in separate equilibrium binding assays. Exemplary such assays are given in Kotani et al. the reaction is stopped by filtration through GF/B filters, using a cell harvester, or by directly measuring the bound ligand. If the ligand was labeled with a radioactive isotope such as ³H, ¹⁴C, ¹²⁵I, ³⁵S, ³²P, ³³P, etc., the bound ligand may be detected by using liquid scintillation counting, scintillation proximity, or any other method of detection for radioactive isotopes. If the ligand was labeled with a fluorescent compound, the bound labeled ligand may be measured by methods such as, but not restricted to, fluorescence intensity, time resolved fluorescence, fluorescence polarization, fluorescence transfer, or fluorescence correlation spectroscopy. In this manner agonist Kiss-1 derived proteins that bind to the orphan receptor may be identified as they inhibit the binding of the labeled ligand to the membrane protein or intact cells expressing the said receptor. Non-specific binding is defined as the amount of labeled ligand remaining after incubation of membrane protein in the presence of a high concentration (e.g., 100-1000×K_{D}) of unlabeled ligand.

In equilibrium saturation binding assays membrane preparations or intact cells transfected with the orphan receptor are incubated in the presence of increasing concentrations of the labeled compound to determine the binding affinity of the labeled ligand. The binding affinities of unlabeled compounds may be determined in equilibrium competition binding assays, using a fixed concentration of labeled compound in the presence of varying concentrations of the displacing ligands. For example, the Kiss-1 derived proteins of the present invention also may be competitive inhibitors of the kisspeptins of SEQ ID NO: 4, 5 or 6 or other naturally occurring ligand of GPCR54 receptor for GPCR54 receptor binding. To measure the relative binding affinities of selected Kiss-1 derived proteins, an ELISA-type approach may be employed. For example, to examine binding affinity for GPCR54 receptor, serial dilutions of competing kisspeptin of SEQ ID NO: 4, 5, or 6 and a subsaturating concentration of the candidate Kiss-1 derived peptide tagged with the myc epitope is added to microtitre plates coated with GPCR54, and incubated until equilibrium is established. The plates are then washed to remove unbound proteins. Peptides that remain bound to the receptor-coated plates are detected using an anti-myc antibody conjugated to a readily detectable label e.g., horseradish peroxidase. Binding affinities (EC₅₀) can be calculated as the concentration of competing Kiss-1 derived protein that results in half-maximal binding. These values can be compared with those obtained from analysis of kisspeptin of SEQ ID NO: 4, 5, or 6 to determine the relative binding affinity for the receptor of the tested Kiss-1 derived protein.

Functional Assays

The Kiss-1 derived proteins may be tested to determine whether the test proteins increase any signaling activity associated with the GPCR54 receptor, thereby determining whether the Kiss-1 derived protein is a GPCR54 receptor agonist. The signaling response may be a second messenger response of the GPCR54 and may include e.g., chloride channel activation, change in intracellular calcium levels, release of inositol phosphate, activation of MAP kinase, change in cAMP levels, release of arachidonic acid or other second messenger response typically associated with a GPCR54.

As an alternative to ligand binding assays, cells expressing the GPCR54 receptor DNA may be used to screen for ligands to the receptor using functional assays. It is well known to those in the art that the over-expression of a G-protein coupled receptor can result in the constitutive activation of intracellular signaling pathways. In the same manner, over-expression of the orphan receptor in any cell line as described above, can result in the activation of the functional responses described below, and any of the assays herein described can be used to screen for the activity of the Kiss-1 derived proteins as both agonist ligands of the GPCR54 orphan receptor. Agonists thus identified will be useful in the therapeutic methods of the invention.

As indicated above, a wide spectrum of assays can be employed to screen for the presence of GPCR receptor ligands. These assays range from traditional measurements of total inositol phosphate accumulation, cAMP levels, intracellular calcium mobilization, and potassium currents, for example; to systems measuring these same second messengers but which have been modified or adapted to be of higher throughput, more generic and more sensitive; to cell based assays reporting more general cellular events resulting from receptor activation such as metabolic changes, differentiation, cell division/proliferation. Exemplary such assays are described in detail in U.S. Patent Application Publication No. US2003/0022839 A1 specifically at paragraphs 0100 through 0135. This document is specifically incorporated in its entirety as teaching methods of determining the receptor bind and functional activity of the Kiss-1 derived proteins of the present invention. Any protein or peptide made as described herein may be tested in an assay such as those described in U.S. Patent Application Publication No. US2003/0022839 to determine whether such a protein or peptide retains its receptor agonist activity. As long as the generated protein or peptide retains an activity in any one or more of such exemplary assays (e.g., elevation of cystolic calcium, or phosphorylation of ERK in transfected CHO cells), it may be used in the present invention.

Methods of Treating Infertility

As described herein throughout it has been discovered that Kiss-1 derived proteins can be used to enhance, stimulate, promote or otherwise increase the release of FSH and/or LH. As such, any Kiss-1 peptide/protein that has an activity that is similar to the activity of a peptide of SEQ ID NO:3 may be used in the treatment of infertility disorders. The infertility disorders that may be treated herein include any disorder that may benefit from an increase in the amount of FSH signal, LH signal or both. Preferably, the increase in FSH production produced by the treatment methods of the invention results in an appreciable increase in the blood levels of FSH. In preferred embodiments, the methods produce a greater than 2-fold increase in blood levels of FSH, however, any increase in FSH is likely to be beneficial. It is contemplated that the peptide/protein-based compositions of the present invention may be used in any and all protocol in which an agent such as Clomiphene is presently be used in the treatment of a fertility disorder. As such, the protein/peptide-based therapeutics of the present invention may be used in the treatment of anovulatory females in the induction of ovulation and pregnancy in anovulatory infertile patients in whom the cause of infertility is functional and not due to primary ovarian failure or individuals suffering from hypogonadotropic hypogonadism. In men these peptides may be useful in the induction of spermatogenesis in males having primary and secondary hypogonadotropic hypogonadism. The peptides also may be useful in the treatment of endometriosis.

The protocols for the administration of the Kiss-1 derived proteins may be similar to the protocols for the administration of other stimulants of gonadotropin release. As a general guideline, protocols developed for the administration of clomiphene may form a starting point for the administration of the peptides of the invention as both clomiphene and the Kiss-1 related peptides are both used to stimulate gonadotropin production. Thus, for example to stimulate ovulation, the Kiss-1 derived protein-based compositions (e.g., a peptide of amino acid sequence of SEQ ID NO:3) may be prescribed for five days each cycle, typically as a single daily dose on each specified day. However, the dosage may be increased or decreased by your physician based on the patient's individual response. In a typical treatment, like clomiphene, it may be necessary to perform a Kiss-1 protein load test determine whether the patient will be responsive to the Kiss-1 derived protein. Clomiphene load tests are well known to those of skill in the art (See e.g., U.S. Pat. No. 5,091,170). Such tests can be modified by replacing the clomiphene with the Kiss-1 derived protein being used as the stimulant.

In a typical protocol for the ovulation stimulation, the Kiss-1 derived protein may be initially administered beginning on cycle day 3 and taken daily until cycle day 7. At cycle day 9 or 10, the LH and FSH levels of the patient are monitored. If the LH level is two to three times higher than the FSH level, the Kiss-1 derived protein may not be effective. On cycle day 12, the urine of the patient is tested daily for LH surge, i.e., the signal that ovulation will occur in 24-36 hours. Ovulation predictor kits (LH predictor) may be used for this test. If a surge has not occurred by cycle day 16, an ultrasound may be performed to check for follicular development and measure the thickness of the uterine lining. After LH surge ovulation should occur with two days.

If pregnancy does not result, a further cycle of Kiss-1 derived protein therapeutic is begun. In such a subsequent cycle, at the onset of menstrual flow, before day three, a pelvic examination and or ultrasound check may be performed.

In alternative embodiments, where the above protocol is unsuccessful in producing a pregnancy, intrauterine insemination may be used to improve possibility of conception. Such intrauterine insemination may be combined with one or more of clomiphene, letrozole, HMG/insemination or Gonal-F/Follistim injections and intrauterine insemination. In intrauterine insemination, a baseline ultrasound is performed on or before cycle day 3. Beginning at day 3 the Kiss-1 derived protein therapy is initiated and continued to cycle day 7. In combined therapies, the patient may be treated with the Kiss-1 derived protein, in combination with clomiphene (or letrozole)/FSH or HMG/and intrauterine insemination. In such protocols, an injection of 150 units of FSH or HMG (e.g., Bravelle/Gonal-F or Repronex) may be administered on day 8 or 9. On cycle 9 or 10 LH and FSH are determined.

If the patient does not have an LH surge by the 16th cycle day, an ultrasound should be performed to check for follicular development and measure the thickness of the uterine lining. In the event that it is determined that the follicle is larger than 19 mm and endometrium is thicker than 7 mm an hCG injection may be administered to trigger release of egg(s).

Additional Compounds/Compositions to be Administered with Kiss-1 Protein

As discussed above, the protocols for the administration of the Kiss-1 derived proteins may be combined with other agents to produce e.g., ovulation stimulation. Thus, while the Kiss-1 derived proteins are contemplated to be sufficient to produce an increase in FSH release, a surge in LH or both of these effects, which may thus be used in improve stimulation of ovulation and overcome infertility, it is contemplated that the Kiss-1 derived proteins also may be administered in combined therapies with other treatments for infertility. Such additional treatments include administration of other stimulators of gonadotropin release e.g., clomiphene and letrozole, as well as various gonadotropins to increase ovulation induction and/or follicle maturation.

To achieve the appropriate therapeutic outcome in the combination therapies contemplated herein, i.e., to achieve an increase in ovulation induction, produce an increase in FSH production, produce an LH surge, produce an increase in the number of ovulatable oocytes in the mammal that is being treated and the like, one would generally administer to the subject the Kiss-1 derived protein composition in an amount effective to produce the desired therapeutic outcome. In addition, additional exogenous FSH may also be provided in a course of daily administrations lasting between 7 to 12 days. FSH compositions are described in further detail below.

FSH is a pituitary glycoprotein hormone that is composed of two subunits. The α-subunit is common to FSH as well as the other glycoproteins, LH, hCG and TSH, the β-subunit confers FSH specificity. The field of infertility treatment is advanced and there are presently numerous FSH preparations that are commercially available and may be used in the methods of the invention. Such commercial preparations include urinary-derived FSH compositions and recombinant FSH compositions. These compositions include, e.g., Pergonal™ (Serono Laboratories Inc., Randolph, Mass.; protocols and compositions for typical administration described in e.g., PDR™, 52^{nd} Edn. 1998, pages 2773-2775), Fertinex™ (Serono Laboratories Inc., Randolph, Mass. protocols and compositions for typical administration described in e.g., PDR™, 52^{nd} Edn. 1998, pages 2771-2773), Repronex™ (Ferring Pharmaceutical Inc., Tarrytown, N.J.; protocols and compositions for typical administration described in PDR™, 57^{th} Edn. 2003, pages 1325-1327), (Ferring Pharmaceutical Inc., Tarrytown, N.J.; described in e.g., PDR™, 57^{th} Edn. 2003, pages 1325-1327), Humegomm (Organon, West Orange, N.J.; protocols and compositions for typical administration described in e.g., PDR™, 52^{nd} Edn. 1998, pages 1949-1951), Gonal-F ((PDR™, 57^{th} Edn. 2003, pages 3124-3128), Follistim™. These are merely exemplary commercial FSH preparations and those of skill in the art will understand that it may be possible to produce other such FSH preparations for use in the methods and compositions of the present invention. To the extent that the preceding compositions provide exemplary guidance as to formulations and dosages of FSH that may be used, they are discussed in further detail below. However, it should be understood that such doses and formulations may readily be modified and still be useful in the context of the present invention as long as the FSH dosages and formulations when administered in combination with Kiss-1 derived proteins produce a therapeutically effective LH surge and/or an increase in the number of ovulatable oocytes in vivo as compared to the number of oocytes produced in the absence of such administration.

In addition to these commercially available compositions, those of skill in the art may chose to purify FSH from natural source, e.g., urine of post-menopausal women, using techniques well known to those of skill in the art (See e.g., U.S. Pat. No. 5,767,067).

Alternatively, those of skill in the art may choose to produce recombinant FSH using techniques well known to those of skill in the art. It is particularly contemplated that long-lasting FSH agonists would be useful in the methods of the invention. For example, it is known that hCG has a longer half life than FSH. Both of these gonadotropins share a common α-subunit, with the specific activity being conferred by the β-subunit. It has previously been demonstrated that the α-subunit of one gonadotropin may be used with the β-subunit of another and still yield a physiologically active chimeric gonadotropin. Further it has been demonstrated that the increased biopotency of hCG as compared to LH was due to the carboxy-terminal peptide of the β-subunit of hCG (Matzuk et al., Endocrinology 126:376-383, 1990). Long lasting agonists of FSH may be generated which contain a carboxy-terminal peptide extension of hCG β-subunit at the carboxy terminus of the FSH b subunit. (LaPolt et al., Endocrinology, 131:6, 2514-2520, 1992). Such chimeric molecules have been shown to possess a markedly increased circulating half life and potency as compared to wild-type FSH (Fares et al., Proc. Nat'l Acad. Sci., 89:4304-4308, 1992). Longer half-life can also be achieved by extra glycosylations or PEGylations as described elsewhere in the present application.

The FSH may be administered through any route normally employed for the administration of gonadotropin hormones. Most preferably the administration is either via intramuscular or subcutaneous injection. Throughout the treatment protocols, the patient is monitored for signs of adverse reaction including for signs of OHSS.

In addition to FSH, other gonadotropin hormones will be used in the methods of the present invention and packaged in the kits described herein. Such hormones include hCG. This is commercially available as Novarel™ (Ferring Pharmaceutical Inc., Tarrytown, N.J.), described in the Physician's Desk Reference (see e.g., PDR™, 57^{th} Edn. 2003, pages 1324-1325) and is a gonadotropin produced by the human placenta and obtained from the urine of pregnant women. Another commercial preparation of hCG is Pregnyl™ (Organon, West Orange, N.J.). The properties, indications and protocols for the use of this hormone are discussed in detail in the Physician's Desk Reference. (see e.g., PDR™, 57^{th} Edn. 2003, pages 2401). Both of these preparations are for intramuscular administration. Typically, this hormone is administered in a dosage of between about 5000 Units and 10 000 Units to induce ovulation.

Yet another hormone that may be used and packaged herein is GnRH. There are numerous commercial sources of this hormone. GnRH and analogs thereof are commercially available as Cetrotide™ (Serono; see PDR™, 57^{th} Edn. 2003, pages 3119-3121); Eligard™ (Sanofi-Synthelabo, PDR™, 57^{th} Edn. 2003, page 2994); Lupron™ (PDR™, 57^{th} Edn. 2003, page 3185); and Zoladex™ (AstraZeneca PDR™, 57^{th} Edn. 2003, page 695). These agents are used to suppress LH/FSH production in women and are therefore used to delay ovulation. Typical doses of these agents vary from about 0.25 mg to about 3 mg. Ovarian stimulation therapy with FSH is typically initiated on the 2^{nd} or 3^{rd} day of the menstrual cycle. The GnRH or analogs thereof are administered either once daily (lower dose, e.g., 0.25 mg), or as a single dose (e.g., 3 mg) during the early to mid follicular phase. GnRH is administered up until the day of hCG administration. When ultrasound analyses reveal that the follicles are of an adequate size, hCG is administered to induce ovulation and final maturation of the oocyte.

In the other embodiments in which there are multiple therapeutic agents that are co-administered, the co-administered agents may be administered concurrently with one another or may be administered separately after appropriate time intervals. For example, clomiphene may be administered at the same time as the Kiss-1 derived protein-based compositions. Commercial preparations of clomiphene are known to those of skill in the art and include e.g., Clomid™ and Serophene™. In other embodiments, the Kiss-1 derived protein may be coadministered with a GnRH antagonist. Such GnRH antagonists, e.g., Lupron®, Synarel®, Antigon®, or Cetrotide® and protocols for their administration in fertility treatment are well known to those of skill in the art and are readily commercially available.

Patient Selection and Monitoring

The patients that receive the treatments of the invention are female patients between the age range of 20 to 45 year. Patient selection for the methods of the present invention may employ the same parameters as described in the PDR™ entries for use of FSH based therapies described above. For example, prior to treatment the patient is subjected to a thorough gynecologic examination and endocrinologic evaluation, including an assessment of pelvic anatomy. Primary ovarian failure should be excluded by determining the basal serum gonadotropin levels and it should be ensured that the patient is not pregnant.

Throughout the treatment regimens of the present invention, the patient should be assessed prior to, during, and after, the therapy to monitor for the signs of OHSS. The symptoms of OHSS include but are not limited to abdominal pain, abdominal distention, gastrointestinal symptoms including nausea, diarrhea, severe ovarian enlargement, weight gain, dyspnea and oliguria. Clinically, the symptom manifests in hypovolemia, hemoconcentration, electrolytic imbalance, ascites, hemoperitoneum, pleural effusions, hydrothorax acute pulmonary distress and thromboembolism. In the event that symptoms of OHSS occur during the administration of the FSH-based therapy or any other agent being administered for stimulation of follicular maturation, the administration should cease and the subject should be placed under medical supervision to determine whether hospitalization or other intervention is necessary. Other symptoms that may be used to monitor the FSH-based therapy include changes in vaginal cytology, appearance and volume of vaginal mucous, Spinnbarkeit and ferning of cervical mucus. These latter symptoms are indicative of the estrogenic effect of the therapy, and should be monitored because administration FSH will stimulate estrogen production. Preferably these estrogenic effects should be monitored in conjunction with more direct determinations of follicle development such as, e.g., determination of serum estradiol and ultrasonigraphy.

The clinical manifestations of ovulation, other than pregnancy, may be obtained either through a direct or an indirect measure of progesterone production. Such indicia include: a rise in basal body temperature, increase in serum progesterone, menstruation following a shift in body temperature. In conjunction with the above indicators of progesterone production, sonographic visualization of the ovaries may be used to assist in determining if ovulation has occurred. Such monographic monitoring may include evaluating fluid in the cul-de sac, ovarian stigmata and the presence of collapsed follicles. Sonographic determinations also will assist in determining whether the ovaries are enlarged in OHSS.

Pharmaceutical Compositions

Pharmaceutical compositions for administration according to the present invention can comprise at least one Kiss-1 derived protein (e.g., a peptide of SEQ ID NO:3, a variant or analog thereof or any other Kiss-1 derived protein that stimulates FSH production and/or release). The pharmaceutical compositions also may include another stimulant of FSH production, e.g., clomiphene and/or at least one gonadotropin hormone, preferably FSH. Each of these preparation is preferably provided in a pharmaceutically acceptable form optionally combined with a pharmaceutically acceptable carrier. These compositions can be administered by any means that achieve their intended purposes. Individualized amounts and regimens for the administration of the compositions for the stimulation of follicle maturation using the methods of the present invention can be determined readily by those with ordinary skill in the art using the guidance provided by the Physician's Desk Reference for the use of such compositions in treating anovulatory disorders and for their use in assisted reproduction technologies. As discussed above, those of skill in the art could initially employ amounts and regimens of FSH, clomiphene and the like currently being used in such medical contexts. To this effect, those skilled in the art are specifically referred to each of the entries in the Physician's Desk Reference discussed above and those entries are incorporated herein by reference in their entireties for teaching methods and compositions for the administration of agents such as Serophene™, Clomid™, Fertinex™, Gonal F™, Bravelle™ and the like discussed herein above. Each of those entries in the Physician's Desk Reference provide exemplary guidance as to types of formulations, routes of administration and treatment regimens that may be used in administering FSH. Any of the protocols, formulations, routes of administration and the like described therein can readily be modified for use in the present invention.

Compositions within the scope of this invention include all compositions comprising at least one Kiss-1 derived protein according to the present invention in an amount effective to achieve its intended purpose of stimulating, increasing or otherwise inducing ovulation. Preferably such induction is preceded by an increase in FSH production and/or an increase in LH surge as compared to these parameters in the absence of administration of these pharmaceutical proteins. In some instances, it is contemplated that the number of ovulatable oocytes in the animal are increased as a result of administration of these peptides, either when administered alone or more preferably, when administered in combination with a low dose of FSH. The active agents used in the methods of the present invention may be administered by any means normally employed for such administration. Most preferably, the Kiss-1 derived protein compositions used in the present invention are administered orally.

It is understood that the suitable dose of a composition according to the present invention will depend upon the age, health and weight of the recipient, kind of concurrent treatment, if any, frequency of treatment, and the nature of the effect desired. However, the most preferred dosage can be tailored to the individual subject, as is understood and determinable by one of skill in the art, without undue experimentation. This typically involves adjustment of a standard dose, e.g., reduction of the dose if the patient has a low body weight. Therapy should be halted in the event that symptoms of OHSS are observed.

As discussed above, the total dose required for each treatment may be administered in multiple doses or in a single dose. The compositions may be administered alone or in conjunction with other therapeutics directed to the disease or directed to other symptoms thereof.

The compositions of the invention should be formulated into suitable pharmaceutical compositions, i.e., in a form appropriate for in vivo applications in a the therapeutic intervention of infertility disorders. Generally, this will entail preparing compositions that are essentially free of pyrogens, as well as other impurities that could be harmful to humans or animals, preferably for oral administration. The FSH formulations may be formulated akin to the currently available FSH preparations discussed herein throughout. The peptide/protein formulations may be formulated similarly to any other small protein composition. Preferably, these formulations are for oral administration, however, other routes of administration are contemplated (e.g. injection, intrathecal administration and the like). The results of the FSH stimulating properties of Kisspeptin lead to a conclusion that a main site of Kisspeptin action is likely in the central nervous system. Such a site of action may be within the blood brain barrier (BBB). As such, it is contemplated that formulations and routes of administration that facilitate the peptide/protein compositions to readily traverse the traverse BBB will be particularly useful. Receptor-mediated uptake across the BBB may be especially useful.

One will generally desire to employ appropriate salts and buffers to render the compositions stable and allow for uptake of the compositions at the target site. Generally the hormone compositions of the invention are provided in lyophilized form to be reconstituted prior to administration and the Kiss-1 derived protein compositions are likely formulated into tablet form. Buffers and solutions for the reconstitution of the hormones may be provided along with the pharmaceutical formulation to produce aqueous compositions of the present invention for administration. Such aqueous compositions will comprise an effective amount of each of the therapeutic agents being used, dissolved or dispersed in a pharmaceutically acceptable carrier or aqueous medium. Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the therapeutic compositions, its use in therapeutic compositions is contemplated. Supplementary active ingredients also can be incorporated into the compositions.

The active compositions of the present invention include classic pharmaceutical preparations of FSH, which have been discussed herein as well as those known to those of skill in the art. Methods of formulating proteins and peptides for therapeutic administration also are known to those of skill in the art. Administration of these compositions according to the present invention will be via any common route so long as the target tissue is available via that route. Most commonly, these compositions are formulated for oral administration. However, other conventional routes of administration, e.g., by subcutaneous, intravenous, intradermal, intramusclar, intramammary, intraperitoneal, intrathecal, intraocular, retrobulbar, intrapulmonary (e.g., term release), aerosol, sublingual, nasal, anal, vaginal, or transdermal delivery, or by surgical implantation at a particular site also may be used particularly when oral administration is problematic. The treatment may consist of a single dose or a plurality of doses over a period of time.

The active compounds may be prepared for administration as solutions of free base or pharmacologically acceptable salts in water suitably mixed with a surfactant, such as hydroxypropylcellulose. Dispersions also can be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients also can be incorporated into the compositions.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups also can be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration.

“Unit dose” is defined as a discrete amount of a therapeutic composition dispersed in a suitable carrier. Parenteral administration of the therapeutic compounds may be carried out with an initial bolus followed by continuous infusion to maintain therapeutic circulating levels of drug product. Those of ordinary skill in the art will readily optimize effective dosages and administration regimens as determined by good medical practice and the clinical condition of the individual patient.

The frequency of dosing will depend on the pharmcokinetic parameters of the agents and the routes of administration. The optimal pharmaceutical formulation will be determined by one of skill in the art depending on the route of administration and the desired dosage. See for example Remington's Pharmaceutical Sciences, 18th Ed. (1990, Mack Publ. Co, Easton Pa. 18042), incorporated herein by reference. Such formulations may influence the physical state, stability, rate of in vivo release and rate of in vivo clearance of the administered agents. Depending on the route of administration, a suitable dose may be calculated according to body weight, body surface areas or organ size. Further refinement of the calculations necessary to determine the appropriate treatment dose is routinely made by those of ordinary skill in the art without undue experimentation, especially in light of the dosage information and assays disclosed herein as well as the pharmcokinetic data observed in animals or human clinical trials.

Appropriate dosages may be ascertained through the use of established assays for determining blood levels in conjunction with relevant dose response data. The final dosage regimen will be determined by the attending physician, considering factors which modify the action of drugs, e.g., the drug's specific activity, severity of the damage and the responsiveness of the patient, the age, condition, body weight, sex and diet of the patient, the severity of any infection, time of administration and other clinical factors. As studies are conducted, further information will emerge regarding appropriate dosage levels and duration of treatment for specific diseases and conditions.

It will be appreciated that the pharmaceutical compositions and treatment methods of the invention may be useful in fields of human medicine and veterinary medicine. Thus the subject to be treated may be a mammal, preferably human or other animal. For veterinary purposes, subjects include for example, farm animals including cows, sheep, pigs, horses and goats, companion animals such as dogs and cats, exotic and/or zoo animals, laboratory animals including mice rats, rabbits, guinea pigs and hamsters; and poultry such as chickens, turkey ducks and geese.

The present invention also contemplated kits for use in the treatment of fertility disorders. Such kits include at least a first composition comprising the proteins/peptides described above in a pharmaceutically acceptable carrier. Another component may be an FSH in a pharmaceutically acceptable carrier. The kits may additionally comprise solutions or buffers for effecting the delivery of the first and second compositions. The kits may further comprise additional compositions which contain further stimulators of FSH production/release e.g., additional other Kiss-1 derived proteins, other stimulators, e.g., clomiphene and/or further hormones such as e.g., hCG, LH and the like. The kits may further comprise catheters, syringes or other delivering devices for the delivery of one or more of the compositions used in the methods of the invention. The kits may further comprise instructions containing administration protocols for the therapeutic regimens.

### EXAMPLES

The following example(s) is included to demonstrate preferred embodiments of the invention. It should be appreciated by those of skill in the art that the techniques disclosed in the example(s) that follows represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1 Exemplary Materials and Methods

Animals

All experiments were conducted in female Sprague-Dawley rats (180-200 g) obtained from Charles River (MA), fed with standard laboratory chow and water ad libitum, kept on 12 h light-dark in temperature and humidity controlled room.

Surgery

All surgeries were conducted under anesthesia with single injection (i.p.) of a mixture of ketamine (100 mg/kg) and xylazine (15 mg/kg).

Implantation of intracerebroventricular cannula for i.c.v. experiment. The head of anesthetized rats was placed in the stereotaxic instrument and oriented such that the bregma and lambda was horizontal. An incision was made along the sagittal midline in an anterior-to-posterior direction. A hole, 1.5 mm in diameter, was drilled through the skull 0.9 mm posterior and 0.0 mm lateral to bregma. A 25 mm×22 gauge stainless steel cannula was lowered about 7 mm until cerebrospinal fluid could be aspirated from the 3rd ventricle via a 1 cc syringe connected to the cannula by a 5 cm section of Silastic tubing. After sealing the outer opening of the cannula, it was anchored to the skull with three stainless steel screws and dental acrylic cement then the incision was closed. Rats were allowed to recover for 14 days.

Ovariectomy and implantation of estrogen-containing capsule. To remove daily variance of plasma estrogen levels due to variable estrous cycle, both ovaries were removed under anesthesia through incision at the animal's back. Right before closure of the incision, two estrogen-containing capsules (0.5 mg/coupsule, Innovative Research of America, FL) were inserted subcutaneously at the middle of animal's back to maintain consistent basal physiological levels of plasma estrogen. Rats were allowed to recover for 7 days.

Jugular cannula insertion and tethering to automated blood sampler. The right jugular vein of anesthetized rats was exposed through a neck incision. A retaining suture was tied to the exposed vein at rostral end to manipulate the vein. The caudal end of the vein was clamped with a small forceps to prevent bleeding while a cannula, a polyethene tubing (OD 1.0 mm), was inserted in the middle of the vein through a 0.5 mm incision made with iris scissors. After the insertion of the cannula, a suture was tied around the vein at the caudal end to secure the cannula within the vein. The incision at the neck was closed with wound clips and the outer end of the cannula was attached to a tubing of an automated blood sampler (Bioanalytical Systems Inc., IN) filled with heparin-containing (20 IU/ml) sterile saline. The automated blood sampler consisting of a turning cage equipped with a food box and a water bottle, a balanced arm, a pumping system, a fraction collector and a controller was used to collect blood samples. A rat inserted of a jugular cannula was tethered to the balanced arm via a loosely-fitting plastic collar. The cage rotates to the counter direction of animal's movement to prevent twisting of the jugular cannula connected to a tubing of the sampler, as well as to allow the rat to eat, drink and move freely during the course of the blood collection. Rats were allowed to recover for 24 hours.

Test Compounds

GnRH, a positive control, Antide, an antagonist of GnRH receptor, and Kiss-1 (45-54; Tyr-Asn-Trp-Asn-Ser-Phe-Gly-Leu-Arg-Phe-NH2; SEQ ID NO:3) was obtained from Sigma (MO), Serono (MA) and Peptide International (KY), respectively. This 10-amino acid fragment of Kiss-1 was used merely because previously it was reported that the fragment showed the most potent binding affinity to GPCR54 (Kotani et al., J Biol Chem 276:34631-34636). Any other Kisspeptin that has receptor binding activity may be similarly used in the methods described herein.

Experimental Procedure

All experiments were carried out around between 13 h and 14 h. GPCR54 agonist, Kiss-1, dissolved in phosphate-buffered saline was injected i.v. and i.c.v. through jugular and intracerebroventricular cannula in a volume of 200 and 10 ul, each. Blood samples (100 ul) were removed before (−15 and 0 min) and after (15, 30, 45 min) injection with the automated blood sampler. Each blood sample was replaced with an equivalent volume of the heparin-containing (20 IU/ml) sterile saline. Plasma was extracted and stored at −20° C. until determination of LH and FSH by enzyme-linked immunoassay and radioimmunoassay, respectively.

Data Analysis

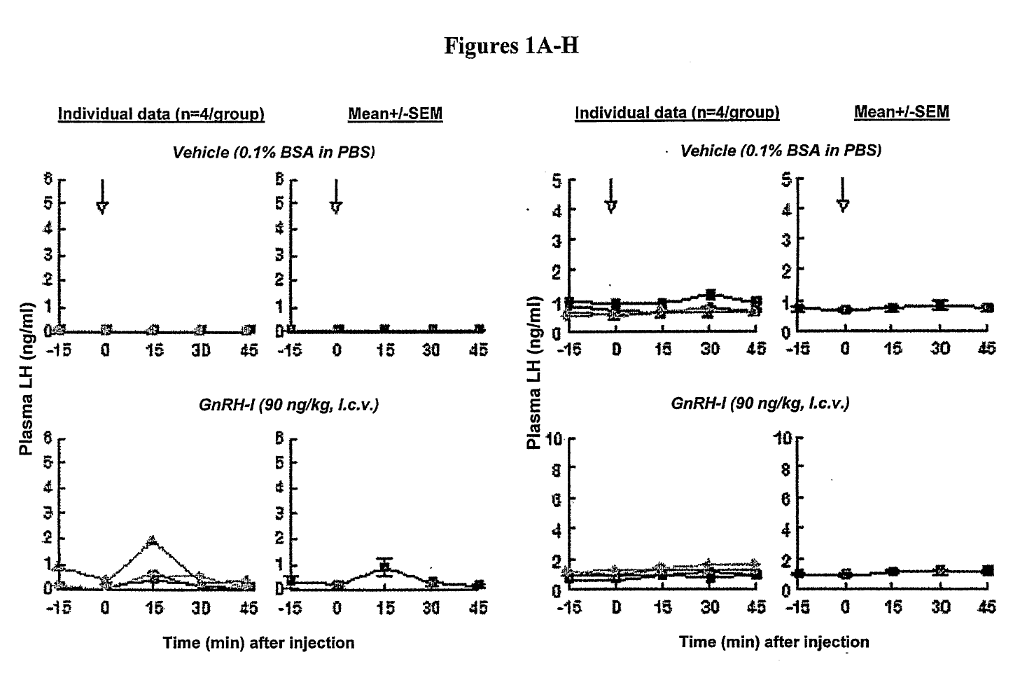

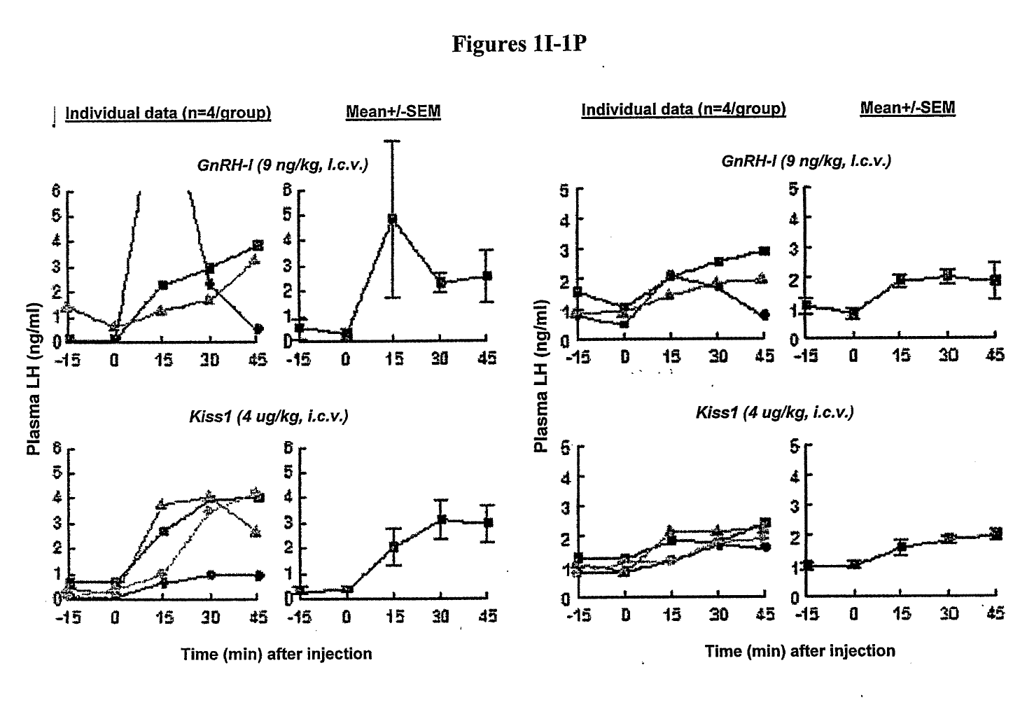

Changes of plasma LH and FSH concentration (ng/ml) after injection were obtained as the area under the curve (AUC). It was first determined for each animal by calculating the sum of post-treatment hormonal levels at 15, 30 and 45 min each of which was subtracted by basal levels (an average of pre-treatment levels at −15 and 0 min), then averaged as a group. Statistical differences among treatment groups were determined by one-way ANOVA followed by LSD as a post-hoc test. An initial data set is shown in FIGS. 1A-P. Further data are described in Examples 2 and 3 below.

### Example 2 Stimulation of the Gonadotropic Axis by i.v. Injection of Kiss-1

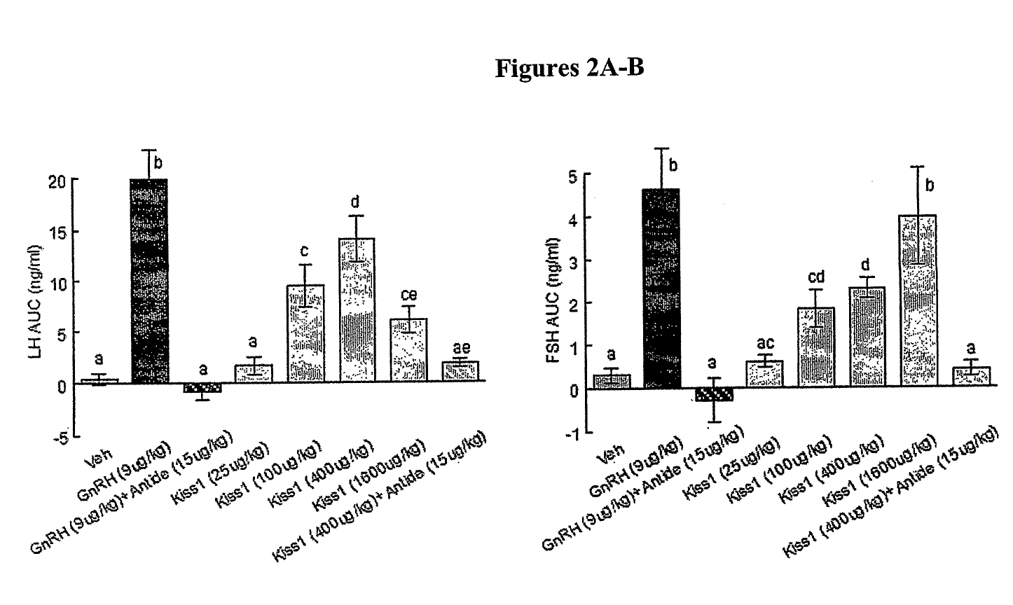

This study aimed to determine the effects of peripheral treatment of Kiss-1 on LH and FSH release. It also determined if the effects of Kiss-1 was mediated by GnRH receptor in the pituitary, a well-established mechanism in the regulation of gonadotropins, by the pretreatment of GnRH receptor antagonist, Antide, 15 min before the injection of 400 ug/kg of Kiss-1. These results were summarized in FIG. 2. There were dose-dependent increases by Kiss-1 treatment in plasma LH and FSH levels with the exception of LH response at the highest dose. A blockade of GnRH receptor successfully prevented GnRH- and Kiss-1-induced elevations of circulating gonadotropins levels. These results demonstrate that stimulation of the gonadotropic axis by Kiss-1 is mediated by GnRH receptor in the pituitary.

### Example 3 Stimulation of the Gonadotropic Axis by i.c.v. Injection of Kiss-1

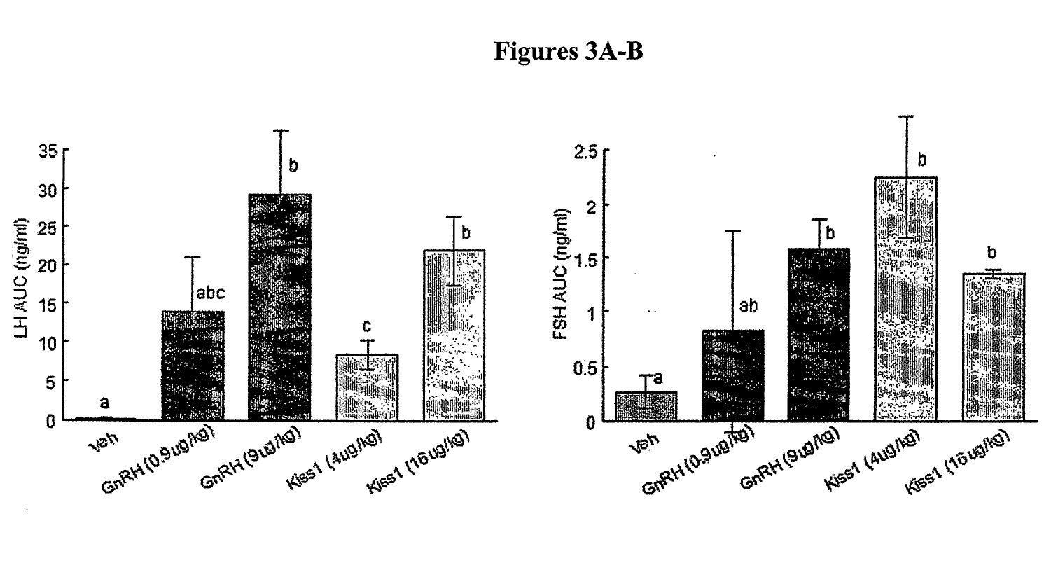

Based on the observation that GnRH antagonist prevents stimulatory effects of Kiss-1 on LH and FSH release as shown in Example 2, it was hypothesized that Kiss-1 acts in the brain, where GnRH neurons locate, to stimulate their neural activity. To test this hypothesis, this study was aimed to determine if i.c.v. injection of Kiss-1 stimulates the gonadotropic axis. These results were summarized in FIG. 3. Kiss-1 dose-dependently stimulated LH release. Further, while the higher dose of Kiss-1 was not as effective as the lower dose, there was a significant increase of FSH levels after Kiss-1 injection. It is worthwhile to mention that i.c.v. doses of Kiss-1 to yield responses of LH and FSH in i.v. injection study were as low as 1/25 and 1/100 of i.v. doses, respectively. These results demonstrate that main action site of Kiss-1 in enhancing LH and FSH release is in the brain and the regulatory mechanism could involve stimulation of GnRH neurons.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

The references cited herein throughout, to the extent that they provide exemplary procedural or other details supplementary to those set forth herein, are all specifically incorporated herein by reference.

What is claimed is:

1. A method of stimulating ovulation in an mammal comprising administering to said mammal a first composition comprising a Kiss-1 derived protein.

2. The method of claim 1, wherein said Kiss-1 derived protein is a mature Kiss-1 protein comprising the sequence of SEQ ID NO:2.

3. The method of claim 1, wherein said Kiss-1 derived protein is a peptide derived from a mature Kiss-1 protein.

4. The method of claim 1, wherein said Kiss-1 derived protein is a Kiss-1 derived peptide that is able to activate a GPCR54 receptor.

5. The method of claim 1, wherein said Kiss-1 derived protein is a peptide that comprises an amino acid sequence of SEQ ID NO:7.

6. The method of claim 1, wherein said Kiss-1 derived protein is a peptide having the sequence of having the sequence of SEQ ID NO:3, an analog of a peptide of SEQ ID NO:3, a fragment of SEQ ID NO:3, or an analog of a fragment of SEQ ID NO:3.

7. The method of claim 6, wherein said analog of SEQ ID NO:3 is a peptide that is a conservative variant of SEQ ID NO:3.

8. The method of claim 1, further comprising administering a second composition that stimulates ovulation.

9. The method of claim 8, wherein said second composition that stimulations ovulation comprises a hormonal or a chemical stimulant of ovulation.

10. The method of claim 9, wherein said chemical stimulant of ovulation is clomiphene citrate or Letrazole.

11. The method of claim 9, wherein said hormonal stimulant of ovulation is a gonadotropin hormone selected from the group consisting of human menopausal gonadotropin (hMG), follicle stimulating hormone (FSH), luteinizing hormone (LH), and human chorionic gonadotropin (hCG).

12. The method of claim 11, further comprising administering a composition comprising GnRH antagonist.

13. The method of claim 11, wherein said gonadotropin is FSH.

14. The method of claim 13, further comprising administering a non-FSH gonadotropin hormone.

15. The method of claim 14, wherein said FSH and said LH are administered in equal amounts.

16. The method of claim 13, wherein said FSH is administered at a dosage range of from about 5 to 450 IU/day.

17. The method of claim 13, wherein said FSH is administered at a dosage range of from about 5 to 75 IU/day

18. The method of claim 11, wherein said gonadotropin hormone is administered by injection.

19. The method of claim 1, wherein said mammal is a human.

20. A method of stimulating FSH production in a mammal comprising administering to said mammal a composition comprising a Kiss-1 derived protein.

21. The method of claim 20, wherein said Kiss-1 derived protein is a mature Kiss-1 protein comprising the sequence of SEQ ID NO:2.

22. The method of claim 20, wherein said Kiss-1 derived protein is a peptide derived from a mature Kiss-1 protein.

23. The method of claim 20, wherein said Kiss-1 derived protein is a Kiss-1 derived peptide that is able to activate a G-protein coupled receptor 54.

24. The method of claim 20, wherein said Kiss-1 derived protein is a peptide that comprises an amino acid sequence of SEQ ID NO:7.

25. The method of claim 20, wherein said Kiss-1 derived protein is a peptide having the sequence of SEQ ID NO:3, an analog of a peptide of SEQ ID NO:3, a fragment of SEQ ID NO:3, or an analog of a fragment of SEQ ID NO:3.

26. The method of claim 25, wherein said analog of SEQ ID NO:3 is a peptide that is a conservative variant of SEQ ID NO:3.

27. The method of claim 20, further comprising administering a second composition that stimulates FSH production in said mammal.

28. The method of claim 27, wherein said second composition that stimulated FSH production comprises an anti-estrogenic compound.

29. The method of claim 28, wherein said anti-estrogenic compound is selected from the group consisting of clomiphene and letrazole.

30. A method of stimulating LH production in a mammal comprising administering to said mammal a composition comprising a Kiss-1 derived protein.

31. A method for the therapeutic intervention of infertility in a mammal comprising:
a) suppressing endogenous gonadotropins production in said mammal,
b) administering to said mammal a composition Kiss-1 related peptide in an amount effect to stimulate ovarian follicle growth in said mammal; and
c) inducing ovulation in said mammal to produce an ovum.

32. The method of claim 31, wherein said method further comprises administering a gonadotropin preparation to stimulate ovarian follicle growth.

33. The method of claim 32, wherein said gonadotropin preparation comprises urinary or recombinant FSH or HMG, with or without recombinant LH.

34. The method of claim 32, wherein said gonadotropin preparation comprises recombinant FSH.

35. The method of claim 31, wherein said inducing ovulation comprises administering to said mammal a composition comprising HCG, native LHRH, LHRH agonists or recombinant LH.

36. The method of claim 31, wherein said suppressing endogenous gonadotropin production in said mammal comprises administering a GnRH antagonist.

37. The method of claim 31, further comprising intrauterine insemination of said ovum by sperm injection.

38. The method of claim 31, wherein said Kiss-1 derived peptide is administered in combination with an anti-estrogenic agent in a combined amount effective to stimulate FSH production in said mammal.

39. The method of claim 31, wherein said mammal is non-responsive to standard gonadotropin stimulation of normal ovulation.

40. The method of claim 31, wherein said infertility disorder is hypogonadotropic hypogonadism.

41. The method of claim 31, wherein said infertility disorder is polycystic ovary syndrome.

42. The method of claim 31, further comprising harvesting oocytes 12 days after the initial stimulation of said ovarian follicle production.

43. The method of claim 42, further comprising fertilizing said harvested oocytes in vitro, and culturing said harvested, fertilized oocytes to the 4-8 cell stage.

44. The method of claim 43, further comprising transferring said 4-8 cell stage fertilized oocytes to the uterus of a mammal.

45. The method of claim 43, wherein said fertilized oocytes are transferred to the uterus of the same mammal from which said oocytes were harvested.

46. The method of claim 43, wherein said fertilized oocytes are transferred to the uterus of a different mammal from which said oocytes were harvested.

47. A method of alleviating one or more of the symptoms hypogonadotropic hypogonadism in a subject comprising administering to said individual a therapeutically effective amount of a Kiss-1 derived peptide.

48. The method of claim 47, wherein said subject is a male subject.

49. The method of claim 47, wherein said subject is a female subject.

48. A composition comprising a Kiss-1 derived protein for use in the treating of infertility.

49. A kit for the treatment of infertility, said kit comprising:
a. a first composition comprising a Kiss-1 derived protein in a pharmaceutically acceptable formulation, and
b. a second composition for the treatment of infertility.

50. The kit of claim 49, wherein said second composition for the treatment of infertility comprises an FSH preparation in a pharmaceutically acceptable formulation.

51. The kit of claim 49, wherein said FSH preparation is selected from the group consisting of a urinary FSH preparation and a recombinant FSH.

52. The kit of claim 50, wherein said FSH is human FSH.

53. The kit of claim 50, wherein said FSH is present in a unit dose of between about 5 IU FSH and about 75 IU FSH.

54. The kit of claim 49, further comprising a third composition comprising LH in a pharmaceutically acceptable formulation.

55. The kit of claim 54, wherein said LH is present in a unit dose of between about 75 IU LH and about 150 IU LH.


SEQUENCE LISTING (11 sequences)

SEQ ID NO: 1 - DNA, 725 bp, Homo sapiens
ctcttgagaccgggagcccagctgcccaccctctggacattcacccagccaggtggtctcgtcacctcagaggctccgccagactcctgcccaggccaggactgaggcaagcctcaaggcacttctaggacctgcctcttctcaccaagatgaactcactggtttcttggcagctactgcttttcctctgtgccacccactttggggagccattagaaaaggtggcctctgtggggaattctagacccacaggccagcagctagaatccctgggcctcctggcccccggggagcagagcctgccgtgcaccgagaggaagccagctgctactgccaggctgagccgtcgggggacctcgctgtccccgccccccgagagctccgggagcccccagcagccgggcctgtccgccccccacagccgccagatccccgcaccccagggcgcggtgctggtgcagcgggagaaggacctgccgaactacaactggaactccttcggcctgcgcttcggcaagcgggaggcggcaccagggaaccacggcagaagcgctgggcggggctggggcgcaggtgcggggcagtgaacttcagaccccaaaggagtcagagcatgcggggcgggggcggggggcggggacgtagggctaagggagggggcgctggagcttccaacccgaggcaataaaagaaatgttgcgtaactcaaaaaaaaaaaaaaaaaa

SEQ ID NO: 2 - protein, 145 aa, Homo sapiens
MNSLVSWQLLLFLCATHFGEPLEKVASVGNSRPTGQQLESLGLLAPGEQSLPCTERKPAATARLSRRGTSLSPPPESSGSPQQPGLSAPHSRQIPAPQGAVLVQREKDLPNYNWNSFGLRFGKREAAPGNHGRSAGRGWGAGAGQ

SEQ ID NO: 3 - protein, 10 aa, Homo sapiens
YNWNSFGLRF

SEQ ID NO: 4 - protein, 54 aa, Homo sapiens
GTSLSPPPESSGSPQQPGLSAPHSRQIPAPQGAVLVQREKDLPNYNWNSFGLRF

SEQ ID NO: 5 - protein, 14 aa, Homo sapiens
DLPNYNWNSFGLRF

SEQ ID NO: 6 - protein, 13 aa, Homo sapiens
LPNYNWNSFGLRF

SEQ ID NO: 7 - protein, 5 aa, Homo sapiens
FGLRF

SEQ ID NO: 8 - protein, 6 aa, Homo sapiens
SFGLRF

SEQ ID NO: 9 - protein, 7 aa, Homo sapiens
NSFGLRF

SEQ ID NO: 10 - protein, 8 aa, Homo sapiens
WNSFGLRF

SEQ ID NO: 11 - protein, 9 aa, Homo sapiens
NWNSFGLRF